(19) United States
(12) Patent Application Publication
(10) Pub. No.: US 2010/0158874 A1
(43) Pub. Date: Jun. 24, 2010

(54) Compositions and Methods for Treating Peripheral Vascular Diseases

(75) Inventors: Ian Rogers, Toronto (CA); Robert Casper, Toronto (CA); Ryszard Bielecki, Mississauga (CA)

Correspondence Address: HOWSON & HOWSON LLP, 501 OFFICE CENTER DRIVE, SUITE 210, FORT WASHINGTON, PA, 19034, US

(73) Assignee: Mount Sinai Hospital, Toronto (CA)

(21) Appl. No.: 12/528,645

(22) PCT Filed: Feb. 26, 2008
(86) PCT No.: PCT/CA2008/000364

§ 371 Date: Jan. 29, 2010

Related U.S. Application Data
(60) Provisional application No. 60/903,461, filed on Feb. 26, 2007.

(51) Int. Cl. A61K 35/12; A61P 9/10; C12N 5/078; G01N 33/53

(52) U.S. Cl. 424/93.7; 435/325; 435/377; 435/7.24

(57) ABSTRACT

The invention relates to methods for producing endothelial cells, pericytes and/or muscle cells (in particular smooth muscle cells), cell preparations and pharmaceutical compositions comprising the cells or preparations, and the use of the cells, preparations and compositions in research or commercial applications. In aspects, the invention provides a method of treating a patient with a condition involving endothelial cells, endothelial precursor cells, pericytes and/or muscle cells, such as a peripheral vascular disease, comprising administering to the patient endothelial precursor cells, endothelial cells, pericytes and/or muscle cells obtained from multipotent CD45⁺HLA-ABC⁺Lin⁻ cells.

### FIELD OF THE INVENTION

The invention relates to methods for producing endothelial cells, endothelial precursor cells (EPCs), pericytes and/or muscle cells, cell preparations and pharmaceutical compositions comprising the cells or preparations, and the use of the cells, preparations and compositions in research or commercial applications.

### BACKGROUND OF THE INVENTION

Peripheral vascular disease (PVD) is due to an organic or functional blockage of the blood vessels similar in mechanism to coronary heart disease. PVD can cause intermittent claudication leading to tissue ischemia of the lower limbs. The prevalence is expected to increase due to the aging population. The unmet medical need for the treatment of intermittent claudication (IC) and critical limb ischemia (CLI) is ˜31 million patients with IC in the US, Europe and Japan; ˜7.8 million require medical treatment and ˜1-5% of IC patients progress to CLI. These patients are at high risk for limb loss and cardiovascular and cerebrovascular complications. The estimated cumulative economic burden is over $30 billion per year in the US alone.

Partial repair of the ischemic tissue can occur due to new vessel formation by (i) angiogenesis and (ii) vasculogenesis or (iii) arteriogenesis. Ischemia acts as a stimulus that causes circulating endothelial precursor cells (EPCs) to home to the site of injury where they proliferate and differentiate into new blood vessels. Standard treatments for PVD are targeted atherosclerotic risk-factor reduction, which generally does not improve tissue perfusion. Therapies to improve tissue perfusion (surgery or angioplasty) target larger vessels and are not generally successful for smaller (peripheral) vessels and limb amputation usually results. Correction of small vessel occlusions and the healing of wounds and skin ulcers require novel therapies. Two significant forays into the therapeutic arena are the use of growth factors to stimulate endogenous cells to undergo vasculogenesis or the transplantation of donor cells. However, pure recombinant growth factors have a short half-life in the body, therefore, the addition of cells capable of secreting factors has the advantage of delivering growth factors in a controlled and sustainable manner.

The citation of any reference herein is not an admission that such reference is available as prior art to the instant invention.

### SUMMARY OF THE INVENTION

The invention provides cell preparations comprising or consisting essentially of endothelial cells and/or endothelial precursors cells (hereinafter collectively referred to as ECs), pericytes and/or muscle cells (particularly smooth muscle cells), obtained from multipotent cells having properties of multipotential mesenchymal cells. The endothelial cells may be characterized by expression of CD31, CD133, Flk-1, von Willebrand factor, and/or VE-cadherin, the pericytes may be characterized by expression of CD31, NG2 chondroitin sulphate proteoglycan, desmin, angiopoietin-1, osteonectin and/or Thy-1, and the muscle cells may be characterized by expression of MyoD, muscle specific actin, Ang-1, PDGF-β and/or myosin heavy chain. ECs, pericytes and/or muscle cells can be isolated and purified from a cell preparation of the invention.

In an aspect, the invention provides cell preparations isolated and cultured in vitro enriched for characteristics of ECs, pericytes, and/or muscle cells. In an embodiment, the invention provides cell preparations isolated and cultured in vitro enriched for characteristics of endothelial cells. In an embodiment, the invention provides cell preparations isolated and cultured in vitro enriched for characteristics of muscle cells, in particular smooth muscle cells.

In an aspect, the invention provides cell preparations comprising endothelial cells and/or muscle cells differentiated in vitro from multipotent cells having properties of multipotential mesenchymal cells and having endothelial cell and/or muscle cell (e.g. smooth muscle cell or striated muscle cell) morphology, respectively, and expressing markers of endothelial cells and/or muscle cells, respectively.

ECs in cell preparations of the invention can have characteristics of endothelial cells or EPCs including one or more of the following: (a) CD31⁺; (b) CD133⁺; (c) Flk-1⁺; (d) elongated cells; (e) capable of growing or ability to grow into a network of vessel-like structures in vitro and in vivo; and (f) capable of secreting or ability to secrete growth factors.

Pericytes in cell preparations of the invention can have characteristics of pericytes including one or more of the following: expression of CD31, NG2 chondroitin sulphate proteoglycan, desmin, angiopoietin-1, osteonectin, and/or Thy-1, and capable of forming or ability to form vessel like structures in vitro and in vivo.

Muscle cells in cell preparations of the invention can have characteristics of muscle cells, in particular smooth muscle cells, including expression of MyoD, muscle actin, and/or myosin heavy chain, and capable of forming or ability to form vessels in vitro and in vivo.

The invention also relates to a system or method for production of cell preparations of the invention comprising culturing multipotent cells having properties of multipotential mesenchymal cells in the presence of one or more differentiation factors or under differentiation conditions to produce a cell preparation comprising EPCs, endothelial cells, pericytes, and/or muscle cells (particularly smooth muscle cells) characterized by one or more of the following properties: (a) EPCs, endothelial, pericyte, and/or muscle cell to morphology; and (b) capable of expressing or ability to express markers of EPCs, endothelial cells, pericytes, and/or muscle cells as the case may be.

In particular aspects of the invention, the multipotent cells may be produced by culturing Lin^{neg} stem and progenitor cells, preferably isolated from umbilical cord blood, under proliferation conditions, in particular in the presence of positive growth factors, more particularly FGF-4, Flt-3 ligand and stem cell factor (SCF), and isolating the multipotent cells in the culture. In a particular aspect, the multipotent cells are CD45⁺HLA-ABC⁺ cells, more particularly CD45⁺HLA-ABC⁺Lin⁻ cells. In aspects of the invention the multipotent cells are enriched for EPCs and/or pericytes. In aspects of the invention the multipotent cells are enriched for endothelial, smooth muscle and/or striated muscle precursor cells. In aspects, the multipotent cells are enriched for muscle and endothelial progenitor cells.

Another aspect of the invention is an enriched or purified cell preparation comprising or consisting essentially of EPCs and/or pericytes produced as described herein.

Another aspect of the invention is an enriched or purified cell preparation comprising or consisting essentially of endothelial cells produced by a method of the invention.

Another aspect of the invention is an enriched or purified cell preparation comprising or consisting essentially of muscle cells, in particular smooth muscle cells, produced by a method of the invention.

Another aspect of the invention is an enriched or purified cell preparation comprising or consisting essentially of ECs, pericytes, and/or muscle cells (particularly smooth muscle cells) produced by a method of the invention.

In an aspect, the invention provides cell preparations comprising ECs (particularly endothelial cells), pericytes, and/or muscle cells (particularly smooth muscle cells) differentiated in vitro from multipotent cells having properties of multipotential mesenchymal cells and wherein the ECs express CD31, CD133, Flk-1, the pericytes express CD31, NG2 chondroitin sulphate proteogly can, desmin, angiopoietin-1, osteonectin, and/or Thy-1, and the muscle cells express MyoD, muscle actin, and/or myosin heavy chain. The cells can have functional features including one or more of the following: (a) the ability to form vessels or stimulate new vessel formation in vitro and in vivo; (b) the ability to stimulate angiogenesis and/or vasculogenesis; (c) the ability to improve blood flow; and (d) the ability to regenerate capillaries (endothelial cells), large vessels (endothelial and smooth muscle cells) and/or striated muscle.

The cell preparations may be used for the preparation of pharmaceutical compositions. Thus the invention also relates to a pharmaceutical composition, in particular a purified pharmaceutical composition, comprising a cell preparation of the invention or EPCs, endothelial cells, pericytes, and/or muscle cells (particularly smooth muscle cells) isolated therefrom, and a pharmaceutically acceptable carrier, excipient or diluent. A pharmaceutical composition may include a targeting agent to target cells to particular tissues or organs.

The invention also contemplates a cell line comprising EPCs, endothelial cells, pericytes, and/or muscle cells (particularly smooth muscle cells) derived from a cell preparation of the invention.

The invention also contemplates cell preparations and pharmaceutical compositions of the invention in combination with a substrate or matrix, preferably a substrate or matrix adapted for transplantation into a patient. The substrate may be an engineered biomaterial or porous tissue culture insert.

The multipotent cells, cell preparations, pharmaceutical compositions and cells therefrom may be used in research or in medical applications. In particular, the multipotent cells, cell preparations and compositions of the invention and cells therefrom can be used in a variety of methods (e.g. transplantation or grafting) and they have numerous uses in the field of medicine. The multipotent cells, cell preparations, compositions and cells therefrom may be used for the replacement of body tissues, organs, components or structures which are missing or damaged due to trauma, age, metabolic or toxic injury, disease, idiopathic loss, or any other cause. The multipotent cells, cell preparations and pharmaceutical compositions comprising the EPCs, endothelial cells, pericytes, and/or muscle cells (particularly smooth muscle cells) or cells therefrom can be used for transplantation to treat a disease disclosed herein including a PVD, more particularly intermittent claudication or critical limb ischemic. In an aspect, the invention provides use of multipotent cells, cell preparations or compositions described herein or cells obtained therefrom for treating a peripheral vascular disease or in the preparation of a medicament for treating such disease. In an aspect of the invention the multipotent cells, cell preparations or compositions of the invention or cells therefrom are used to promote angiogenesis and/or vasculogenesis. In another aspect of the invention the multipotent cells, cell preparations or compositions of the invention or cells therefrom are used to increase vessel diameter (arteriogenesis). In another aspect, the multipotent cells, cell preparations or compositions of the invention or cells therefrom are used to repair ischemic tissue.

The multipotent cells, cell preparations, pharmaceutical compositions or cells therefrom can be used in cell therapies and gene therapies aimed at alleviating disorders and diseases involving EPCs, endothelial cells, pericytes, and/or muscle cells (particularly smooth muscle cells). The invention obviates or reduces the need for human tissue to be used in various medical and research applications.

The invention thus provides a method of treating a patient with a disease or condition involving EPCs, endothelial cells, pericytes, and/or muscle cells (particularly smooth muscle cells), in particular a defect in EPCs, endothelial cells, pericytes, and/or smooth muscle cells, comprising transferring or administering an effective amount of multipotent cells, a cell preparation or pharmaceutical composition of the invention or cells therefrom, optionally with a substrate into the patient. In aspects of the invention, the cell preparations and compositions of the invention are used to treat peripheral vascular disease. In another aspect, the invention provides use of a cell preparation or composition of the invention or EPCs, endothelial cells, pericytes, and/or muscle cells (particularly smooth muscle cells) obtained therefrom for treating peripheral vascular diseases, or in the preparation of a medicament for treating peripheral vascular diseases.

In an aspect, the invention provides a method of treating a patient with a condition involving ischemic tissues comprising:
(a) culturing Lin^{neg} stem and progenitor cells under proliferation conditions to provide multipotent cells wherein the multipotent cells are CD45⁺HLA-ABC⁺ cells;
(b) culturing the mulitpotent cells under suitable differentiation conditions to produce a cell preparation comprising one or more of ECs (particularly endothelial cells) expressing CD31, CD133, and/or Flk-1, pericytes expressing CD31, NG2 chondroitin sulphate proteoglycan, desmin, angiopoietin-1, osteonectin, and/or Thy-1, and/or muscle cells expressing MyoD, muscle actin, and/or myosin heavy chain; and
(c) administering multipotent cells of (a) or the cell preparation of (b) in an effective amount to the patient to treat the condition.

In an aspect, the invention provides a method of treating a patient with a condition involving ischemic tissues comprising:
(a) culturing Lin^{neg} stem and progenitor cells under proliferation conditions to provide multipotent cells wherein the multipotent cells are CD45⁺HLA-ABC⁺ cells and enriched for EPC's and/or pericytes; and
(b) administering multipotent cells of (a) in an effective amount to the patient to treat the condition.

In an aspect, the invention provides a method of treating a patient with a condition involving ischemic tissues comprising:
(a) culturing Lin^{neg} stem and progenitor cells under proliferation conditions to provide multipotent cells wherein the multipotent cells are CD45⁺HLA-ABC⁺ cells;
(b) culturing the mulitpotent cells under suitable differentiation conditions to produce a cell preparation comprising endothelial cells expressing CD31, CD133, and/or Flk-1 and/or muscle cells expressing MyoD, muscle actin, and/or myosin heavy chain; and
(c) administering a cell preparation of (b) in an effective amount to the patient to treat the condition.

The invention also provides a method of treating a mammalian individual suffering from a disease disclosed herein, in particular a peripheral vascular disease comprising: (1) using a method of the invention to obtain multipotent cells or a cell preparation comprising or consisting essentially of EPCs, endothelial cells, pericytes, and/or muscle cells (particularly smooth muscle cells); (2) introducing the multipotent cells or cells from the cell preparation to the mammalian individual, in an amount effective to treat the disease. In particular aspects of the invention the mammalian individual is a human. In other particular aspects the multipotent cells, cell preparation or EPCs, endothelial cells, pericytes and/or muscle cells (particularly smooth muscle cells) therefrom are administered to the mammalian individual by cell transplantation.

Methods of the invention can further comprise co-administering to the mammalian individual a second pharmaceutical composition effective for treating the disease. In particular, an immunosuppressive agent is co-administered with the multipotent cells, cell preparations, cell compositions or cells therefrom.

In an aspect of the invention, multipotent cells, cell preparations and pharmaceutical compositions of the invention are used for autografting, i.e., cells from an individual are used in the same individual. In another aspect, multipotent cells, and cell preparations, pharmaceutical compositions and cells therefrom are used in allografting, i.e., cells from one individual are used in another individual. In a further aspect, the multipotent cells, cell preparations and pharmaceutical compositions and cells therefrom are used for xenografting, i.e., transplantation from one species to another species.

The invention provides a method for obtaining cell preparations or compositions comprising EPCs, endothelial cells, pericytes, and/or muscle cells (particularly smooth muscle cells) for autologous transplantation from a subject's own hematopoietic cells comprising (a) obtaining hematopoietic cells, in particular hematopoietic cells from fresh or cryopreserved umbilical cord blood or bone marrow, from a subject; (b) separating out an enriched cell preparation comprising hematopoietic stem cells and hematopoietic progenitor cells, preferably Lin⁻ stem and progenitor cells; (c) culturing the cells under proliferation conditions, in particular in the presence of FGF4, SCF, and Flt-3 ligand, to produce multipotent cells, more particularly CD45⁺HLA-ABC⁺ cells; and (d) culturing the multipotent cells under suitable culture conditions (e.g., proliferation conditions) or differentiation conditions to produce the cell preparations or compositions. The method may further comprise transferring the cell preparations or compositions to the subject to treat a disease disclosed herein.

In another aspect, the invention provides a method for obtaining cell preparations comprising endothelial cells and/or muscle cells for autologous transplantation from a subject's own hematopoietic cells comprising (a) obtaining hematopoietic cells, in particular hematopoietic cells from fresh or cryopreserved umbilical cord blood or bone marrow, from a subject; (b) separating out an enriched cell preparation comprising hematopoietic stem cells and hematopoietic progenitor cells, preferably Lin⁻ stem and progenitor cells; (c) culturing the cells under proliferation conditions, in particular in the presence of FGF4, SCF, and Flt-3 ligand, to produce multipotent cells, more particularly CD45⁺HLA-ABC⁺ cells; and (d) culturing the multipotent cells under suitable differentiation conditions to produce the cell preparations.

In another aspect, the invention provides a method for obtaining cell preparations comprising pericytes and/or EPCs for autologous transplantation from a subject's own hematopoietic cells comprising (a) obtaining hematopoietic cells, in particular hematopoietic cells from fresh or cryopreserved umbilical cord blood or bone marrow, from a subject; (b) separating out an enriched cell preparation comprising hematopoietic stem cells and hematopoietic progenitor cells, preferably Lin⁻ stem and progenitor cells; and (c) culturing the cells under proliferation conditions, in particular in the presence of FGF4, SCF, and Flt-3 ligand, to produce a cell preparation enriched for pericytes and/or EPCs.

In particular aspects of methods of the invention the hematopoietic cells are cultured under proliferation conditions for at least about 6 to 12 days, 8 to 12 days, 8 to 10 days, preferably about 8 days.

Cell preparations and compositions may be used to screen for potential therapeutics that modulate development or activity of EPCs, endothelial cells, pericytes, and/or muscle cells (particularly smooth muscle cells), and that may be useful in treating peripheral vascular disease. In particular, cell preparations and compositions may be used to screen compounds for an effect on EPCs, endothelial cells, pericytes, and/muscle cells (particularly smooth muscle cells) in which the presence of the compound is correlated with cell maintenance, toxicity, or the ability to function as an EPC, endothelial cell, pericyte, and/or muscle cell (in particular smooth muscle cell or striated muscle cell). Further, the cell preparations and pharmaceutical compositions of the invention and cells therefrom may be used as immunogens that are administered to a heterologous recipient. The cell preparations and pharmaceutical compositions of the invention and cells therefrom may also be used to prepare model systems of disease, or to produce growth factors, hormones, etc.

The invention also relates to a method for conducting a regenerative medicine business. Still further the invention relates to a method for conducting a stem cell business involving identifying agents that affect the proliferation, differentiation, function, or survival of EPCs, endothelial cells, pericytes, and/or muscle cells (particularly smooth muscle cells). An identified agent(s) can be formulated as a pharmaceutical preparation, and manufactured, marketed, and distributed for sale.

In another aspect, the invention contemplates methods for influencing the proliferation, differentiation, or survival of EPCs, endothelial cells, pericytes, and/or muscle cells (particularly smooth muscle cells) by contacting a cell preparation or pharmaceutical composition of the invention or cells therefrom with an agent or agents identified by a method of the invention.

The invention also contemplates a method of treating a patient comprising administering an effective amount of an agent identified in accordance with a method of the invention to a patient with a disorder affecting the proliferation, differentiation, function, or survival of EPCs, endothelial cells, pericytes, and/or muscle cells (particularly smooth muscle cells).

The invention also contemplates a method for conducting a drug discovery business comprising identifying factors or agents that influence the proliferation, differentiation, function, or survival of EPCs, endothelial cells, pericytes, and/or muscle cells (particularly smooth muscle cells), and licensing the rights for further development.

The invention further contemplates a method of providing drug development wherein a cell preparation of the invention or EPCs, endothelial cells, pericytes, and/or muscle cells (particularly smooth muscle cells) in the preparation are used as a source of biological components of EPCs, endothelial cells, pericytes, and/or muscle cells in which one or more of these biological components are the targets of the drugs that are being developed.

The invention also relates to methods of providing a bioassay. The invention also features a kit including multipotent cells, cell preparations or pharmaceutical compositions of the invention. The invention is also directed to a kit for transplantation of EPCs, endothelial cells, pericytes, and/or muscle cells (particularly smooth muscle cells) comprising a flask with medium and multipotent cells, a cell preparation or a pharmaceutical composition of the invention.

The invention also relates to a method of using the cell preparations or compositions of the invention or cells therefrom in rational drug design. In an aspect, the invention relates to a kit for rational drug design comprising a cell preparation or composition obtained by a method of the invention.

These and other aspects, features, and advantages of the present invention should be apparent to those skilled in the art from the following drawings and detailed description.

### DESCRIPTION OF THE DRAWINGS

The invention will now be described in relation to the drawings in which:

FIG. 1 shows an ischemic mouse model. (A) An incision is made in the right hind-limb of a NOD/SCID mouse. The artery is gently dissected from within the muscles and corresponding nerve and vein. (B) The proximal end of the femoral artery close to the Inguinal ligament, and the distal fragment of the saphenous artery are ligated with 8-0 nylon suture. The whole portion of the artery between ligatures is cut and excised, while the branches are obliterated with an electric coagulator. Care is taken not to create any unnecessary mechanical or thermal damage to the surrounding tissues.

FIG. 2 shows the assessment of positive mice for human endothelial cells. After 1-8 weeks post surgery and injection, the mouse is sacrificed and the hind limb muscle tissue is excised, fixed and prepared for immunohistochemistry. (A) A cross-section of the muscle of a treated animal shows positive staining for CD31 specific for human endothelial cells (green). Nuclei stained with DAPI are blue. (B) A higher magnification. Human cells (CD31+) are clearly incorporated into the vessel. The cells surround red blood cells. (C) Two deconvolution microscope images of the same cell taken at different planes demonstrate that the cell wraps around the blood cells forming a capillary.

FIG. 3 shows the assessment of positive mice for human muscle cells. The cross-section of a mouse treated as in FIG. 2 is positive for human smooth muscle positive cells (green). The positively stained cells are pericytes and are located outside the layer of endothelial cells demonstrating that UCB cells are capable of contributing to both the endothelial and muscle cells of large vessels.

FIG. 4 shows in vitro differentiation of Lin-UCB cells grown in Fgf4/SCF/Flt3-ligand: (A) UCB Lin⁻ cells grown first in FGF4/SCF/Flt-3 ligand for 8-days will express Flk1, an embryonic endothelial cell marker. (B) Moving the cells to endothelial differentiation medium resulted in the elongation of the cells and expression of the mature endothelial marker CD31. (C) The FGF4/SCF/Flt-3 ligand grown cells will form primitive capillaries linking separate colonies in a 3-D fibrogen matrix.

FIG. 5 shows production of a hind limb ischemia model in NOD/SCID mice. (A) Surgery exposes the femoral artery which is then ligated. The hind-limb ischemic injury is reproduced by surgical ligation of the femoral artery. Care is required not to nick the vein or nerve. (B) Cross section of muscle post surgery. Note reduction in size of muscle fibres and infiltrating lymphocytes. The localized ischemia is evidenced by the degenerated muscle fibers and infiltration of lymphocytes as observed in the center of the tissue section. The nuclei of the lymphocytes are stained blue.

FIG. 6 shows FSF1 cell engraftment and differentiation in the Hind limb ischemia model (NOD/SCID mouse) and in particular, transplantation of multipotent cells (human UCB cells grown in FSF1 medium) into the adductor and gastrocnemius muscles of the injured leg post-surgery as revealed by immunochemistry. Cross sections of mouse hind limb were stained with human specific antibodies. Mice transplanted with multipotent cells and analyzed for engraftment by immunochemistry at 1 week post-transplantation, stained positive for human CD31, indicating that the multipotent cells fully differentiated in vivo within one week (A; 20×, B; 100×). Mice analyzed 8 weeks post-transplantation remained positive for human cells indicating that the engrafted and differentiated cells can survive long term. Mice analyzed 2 weeks post-transplantation stained positive for human smooth muscle actin (C; 20×) and human muscle actin (D; 20×) indicating the presence of striated muscle cells. Similar results were observed in mice at 4 and 8 weeks, again indicating that the engrafted and differentiated cells can survive long term.

FIG. 7 shows Microcomputed tomography (MicroCT) and laser Dopler Imaging analyses. Analysis of the ischemic limb of a mouse that received FSF1 grown cells. MicroCT and laser Doplet analyses revealed an increased vascular bed and an increased blood flow, respectively, in the injured hind-limb treated with FSF1 grown cells as compared to the untreated control leg. Laser Doppler analyses demonstrated that the animal treated with FSF1 grown cells in the ischemic right leg had blood flow recovery to 73.5% of normal while the control animal (no cells) only recovered to 47% of normal blood flow.

FIG. 8 shows FISH analysis which reveals that human cells produce endothelial cells through differentiation and not fusion of human and mouse cells. FISH analysis of engrafted human cells using human centromeric probes (green) and mouse centromeric probes (red) to detect fused cells. No double positive cells were found confirming that fusion does not occur. The human cells therefore differentiate due to signals from the surrounding tissue.

FIG. 9 shows differentiation of multipotent cells into endothelial cells. The multipotent cells (human UCB cells grown in FSF1 medium) were cultured in endothelial differentiation medium then examined by immunochemistry for the expression of endothelial markers. (a) Prior to culture in differentiation medium the multipotent cells expressed Flk-1. (b) After one week in differentiation medium, the cells expressed the mature endothelial marker CD31. (c) After two weeks in differentiation medium 100% of the cells expressed CD31. (d) After culturing mulitpotent cells in a 3-dimensional fibrin matrix for 3-4 weeks, primitive vessel-like structures could be observed in culture.

FIG. 10 shows differentiation of multipotent cells (human UCB cells grown in FSF1 medium) into muscle cells. (a) The expression of muscle specific actin protein was detectable by immunocytochemistry when the multipotent cell product was differentiated in muscle differentiation medium. The representative result shown is from multipotent cells cultured in reduced serum (1%) and normoxia conditions. (b) Myosin heavy chain expression was observed in the multipotent cell muscle-differentiated cells. Myosin heavy chain was expressed in the muscle cells that had undergone fusion whereas individual cells remained negative for myosin heavy chain.

### DETAILED DESCRIPTION OF PREFERRED EMBODIMENTS

In accordance with the present invention there may be employed conventional molecular biology, microbiology, and recombinant DNA techniques within the skill of the art. Such techniques are explained fully in the literature. See for example, Sambrook, Fritsch, & Maniatis [Sambrook, Fritsch, & Maniatis, Molecular Cloning: A Laboratory Manual, Second Edition (1989) Cold Spring Harbor Laboratory Press, Cold Spring Harbor, N.Y]; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription and Translation [B. D. Hames & S. J. Higgins eds (1984)]; Animal Cell Culture [R. I. Freshney, ed. (1986)]; Immobilized Cells and Enzymes [IRL Press, (1986)]; and B. Perbal, A Practical Guide to Molecular Cloning (1984). The invention may also employ standard methods in immunology known in the art such as described in Stites et al.(Stites et al. (eds) Basic and Clinical Immunology, 8^{th} Ed., Appleton & Lange, Norwalk, Conn. (1994); and Mishell and Shigi (Mishell and Shigi (eds), Selected Methods in Cellular Immunology, W.H. Freeman and Co., New York (1980). Cell culture methods are generally described in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), Embryonic Stem Cells: Methods and Protocols (K. Turksen ed. Humana Press). Tissue culture reagents and materials are commercially available from companies such as Gibco/BRL, Nalgene-Nunc International, Sigma Chemical Co., StemCell Technologies and ICN Biomedicals.

For convenience, certain terms employed in the specification and claims are collected here. Unless defined otherwise, all technical and scientific terms used herein have the same meaning as commonly understood by one of ordinary skill in the art to which this invention belongs.

As used herein, the terms “comprising,” “including,” and “such as” are used in their open and non-limiting sense.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and “the” include plural referents unless the content clearly dictates otherwise. The term “about” means plus or minus 0.1 to 50%, 5-50%, or 10-40%, preferably 10-20%, more preferably 10% or 15%, of the number to which reference is being made.

“Patient”, “subject” or “individual” refers to an animal, preferably a human, to whom treatment, including prophylactic treatment, with the cells, preparations, and compositions of the present invention, is provided. For treatment of those conditions or disease states that are specific for a specific animal such as a human patient, the term refers to that specific animal. Preferably, the terms refer to a human. The terms also include domestic animals bred for food, sport, or as pets, including horses, cows, sheep, poultry, fish, pigs, cats, dogs, and zoo animals. A “donor” refers to an individual (animal, including a human) who or which donates cells, in particular umbilical cord blood for use in a patient.

“Effective amount” refers to concentrations of components such as growth factors, cells, preparations, or compositions effective for producing an intended result including production of cell preparations of the invention, or treating a disease or condition with the cells, cell preparations and pharmaceutical compositions of the invention, or for effecting a transplantation of such cells, cell preparations or pharmaceutical compositions within a to patient to be treated. In the case of administration to a patient, an effective amount can provide a dosage which is sufficient in order for prevention and/or treatment of a condition or disease in the patient compared with no treatment or another treatment.

The terms “administering” or “administration” refers to the process by which multipotent cells, EPCs, endothelial cells, pericytes and/or muscle cells (particularly smooth muscle cells), preparations, or compositions of the invention or cells therefrom, are delivered to a patient for treatment purposes. Cells, preparations, or compositions may be administered a number of ways including parenteral (e.g. intravenous and intraarterial as well as other appropriate parenteral routes), intrathecal, intraventricular, intraparenchymal, intracisternal, intracranial, intrastriatal, oral, subcutaneous, inhalation, transdermal, or intranigral among others. Generally, a route of administration is selected that allows the cells to migrate or target the site where they are needed. Cells, preparations, and compositions of the invention are administered in accordance with good medical practices taking into account the patient's clinical condition, the site and method of administration, dosage, patient age, sex, body weight, and other factors known to physicians.

“Transplanting”, “transplantation”, “grafting” and “graft” are used to describe the process by which cells, preparations, and compositions of the invention are delivered to the site within the patient where the cells are intended to exhibit a favorable effect, such as treating a disease, injury or trauma, or genetic damage or environmental insult to an organ or tissue. Cells, preparations, and compositions may also be delivered in a remote area of the body by any mode of administration relying on cellular migration to the appropriate area in the body to effect transplantation.

The term “pharmaceutically acceptable carrier, excipient or vehicle” refers to a medium which does not interfere with the function or activity of the multipotent cells, EPCs, endothelial cells, pericytes and/or muscle cells (in particular smooth muscle cells), and which is not toxic to the hosts to which it is administered. A carrier, excipient or vehicle includes diluents, binders, adhesives, lubricants, disintegrates, bulking agents, wetting or emulsifying agents, pH buffering agents, and miscellaneous materials that may be needed in order to prepare a particular composition.

The term “treating” refers to reversing, alleviating, or inhibiting the progress of a disease disclosed herein, in particular a Peripheral Vascular Disease, or one or more symptoms of such disease, to which such term applies. Depending on the condition of the subject, the term also refers to preventing a disease disclosed herein, in particular a Peripheral Vascular Disease, and includes preventing the onset of a disease, or preventing the symptoms associated with such a disease. A treatment may be either performed in an acute or chronic way. The term also refers to reducing the severity of a disease (e.g., Peripheral Vascular Disease) or symptoms associated with such disease prior to affliction with the disease. Such prevention or reduction of the severity of a disease prior to affliction refers to administration of multipotent cells, a cell preparation or pharmaceutical composition of the present invention or cells therefrom to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

“Essentially” refers to a population of cells or a method which is at least 20+%, 30+%, 40+%, 50+%, 60+%, 70+%, 80+%, 85+%, 90+%, or 95+% effective, more preferably at least 98+% effective, most preferably 99+% effective. Therefore, a method that enriches for a given cell population, enriches at least about 20+%, 30+%, 40+%, 50+%, 60+%, 70+%, 80%, 85%, 90%, or 95% of the targeted cell population, most preferably at least about 98% of the cell population, most preferably about 99% of the cell population.

“Isolated” or “purified” refers to altered “by the hand of man” from the natural state i.e. anything that occurs in nature is defined as isolated when it has been removed from its original environment, or both. In an aspect, a preparation, population or composition of cells is substantially free of cells and materials with which it may be associated in nature. By substantially free or substantially purified is meant at least 50% of the population are the target cells, preferably at least 70%, more preferably at least 80%, and even more preferably at least 90%, 95% or 99% are free of other cells. Purity of a population or composition of cells can be assessed by appropriate methods that are well known in the art.

“Gene therapy” refers to the transfer and stable insertion of new genetic information into cells for the therapeutic treatment of diseases or disorders. A foreign gene is transferred into a cell that proliferates to introduce the transferred gene throughout the cell population. Therefore, multipotent cells, EPCs, endothelial cells, pericytes and/or muscle cells (in particular smooth muscle cells), cell preparations and compositions of the invention may be the target of gene transfer, since they will produce various lineages which will potentially express the foreign gene.

As used herein, “hematopoietic cells” refers to cells that are related to the production of blood cells, including cells of the lymphoid, myeloid and erythroid lineages. Exemplary hematopoietic cells include hematopoietic stem cells, primordial stem cells, early progenitor cells, CD34⁺ cells, early lineage cells of the mesenchymal, myeloid, lymphoid and erythroid lineages, bone marrow cells, blood cells, umbilical cord blood cells, stromal cells, and other hematopoietic precursor cells that are known to those of ordinary skill in the art. The hematopoietic cells may be obtained from fresh blood, reconstituted cryoperserved blood, or fresh or reconstituted fractions thereof.

The hematopoietic cells (and the cells in the preparations and compositions of the invention) are preferably mammalian cells, more preferably the cells are primate, pig, rabbit, dog, or rodent (e.g. rat or mouse) in origin. Most preferably, the cells are human in origin. The hematopoietic cells may be obtained from a fetus, a child, an adolescent, or an adult.

In aspects of the invention, the mulitpotent cells are derived from bone marrow cells.

In other aspects of the invention the source of the hematopoietic cells is umbilical cord blood (UCB). “Umbilical cord blood” generally refers to blood obtained from a neonate or fetus. In a preferred embodiment, umbilical cord blood refers to blood obtained form the umbilical cord or placenta of newborns. Hematopoietic cells obtained from UCB offer several advantages including less invasive collection and less severe graft versus host (GVH) reaction [Gluckman et al, N. Eng. J. Med 337:373-81, 1993]. The use of umbilical cord blood also eliminates the use of human embryos as a source of embryonic stem cells. Cord blood may be obtained by direct drainage from the cord and/or by needle aspiration from the delivered placenta at the root and at distended veins.

“Multipotent cells” as used herein refers to cells that show at least one phenotypic characteristic of an early stage non-hematopoietic cell (e.g. stem, precursor, or progenitor non-hematopoietic cells), and preferably at least one phenotypic characteristic of an embryonic stem cell. Such phenotypic characteristics can include expression of one or more proteins specific for early stage non-hematopoietic cells, or a physiological, morphological, immunological, or functional characteristic specific for an early stage non-hematopoietic cell or embryonic stem cell [e.g. Oct-4, Nanog, Stage Specific Embryonic Antigen-3 (SSEA3), and/or Stage Specific Embryonic Antigen-4 (SSEA4)].

Multipotent cells can be produced by first obtaining hematopoietic cells and enriching the cells for hematopoietic stem cells and progenitor cells (sometimes referred to herein as “enriched hematopoietic cell preparation”). The term “stem cells” refers to undifferentiated cells that are capable of essentially unlimited propagation either in vitro, in vivo or ex vivo and capable of differentiation to other cell types. “Progenitor cells” are cells that are derived from stem cells by differentiation and are capable of further differentiation to more mature cell types. Negative and positive selection methods known in the art can be used for enrichment of the hematopoietic cells. For example, cells can be sorted based on cell surface antigens using a fluorescence activated cell sorter, or magnetic beads which bind cells with certain cell surface antigens, in particular lineage specific cell surface antigens (e.g. CD2, CD3, CD14, CD16, CD19, CD24, CD56, CD66b, glycophorin A and/or dextran). Negative selection columns can be used to remove cells expressing lineage specific surface antigens. In aspects of the invention, mature blood cells are removed. The enriched hematopoietic cell preparation essentially comprises or consists essentially of Lin⁻ stem and progenitor cells. An enriched hematopoietic cell preparation can be cultured under proliferation conditions (e.g. in the presence of or media comprising a positive growth factor(s), in particular FGF4, SCF, Flt-3 ligand) to produce multipotent cells.

In an aspect of the invention, multipotent cells are characterized as follows: CD45⁺HLA-ABC⁺ cells, more particularly CD45⁺HLA-ABC⁺Lin⁻ cells. A multipotent cell preparation may be enriched or purified and comprise cells that are at least 70%, 80%, 90%, 95%, 98%, or 99% CD45⁺HLA-ABC⁺Lin⁻ cells. In aspects of the invention the multipotent cells have markers associated with EPCs (e.g, Flk1+). In aspects of the invention the multipotent cells have markers associated with pericytes (e.g. desmin+).

“Suitable differentiation conditions” generally refers to the conditions which provide appropriate elements to enable efficient differentiation of multipotent cells to EPCs, endothelial cells, pericytes and/or muscle cells (in particular smooth muscle cells). These conditions include the use of suitable differentiation media. A differentiation medium generally comprises a minimum essential medium plus optional agents such as growth factors, non-essential amino acids, and other agents known in the art. A differentiation medium may contain serum (FCS) or be serum free. Differentiation media are known to persons skilled in the art and are commercially available from companies such as Celprogen (San Pedro, Calif.) and StemCell Technologies (Vancouver, Canada). A differentiation medium can comprise a differentiation factor which induces multipotent cells to endothelial cells or muscle cells as the case may be. For example, a differentiation factor which induces formation of endothelial cells is vascular VEGF.

An “immunosuppressive agent” refers to any agent which inhibits or prevents an immune response. Exemplary immunosuppressive agents are drugs, for example, a rapamycin; a corticosteroid; an azathioprine; mycophenolate mofetil; a cyclosporine; a cyclophosphamide; a methotrexate; a 6-mercaptopurine; FK506; 15-deoxyspergualin; an FTY 720; a mitoxantrone; a 2-amino-1,3-propanediol; a 2-amino-2[2-(4-octylphenyl)ethyl]; propane-1,3-diol hydrochloride; a 6-(3 dimethyl-aminopropionyl) forskolin; interferon and a demethimmunomycin. Alternatively, an immunosuppressive agent is an antibody including without limitation but 124; BTI-322, allotrap-HLA 15 B270; OKT4A; Enlimomab; ABX-CBL; OKT3; ATGAM; basiliximab; daclizumab; thymoglobulin; ISAtx247; Medi-500; Medi-507; Alefacept; efalizumab; or infliximab.

In aspects of the invention the immunosuppressive agent is one or more of dexamethasone, cyclosporin A, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, tegafur), fluocinolone, triaminolone, anecortave acetate, fluorometholone, medrysone and prednislone.

A “disease” or “condition” refers to a disease/disorder/condition involving endothelial cells, EPCs, pericytes and/or muscle cells (in particular smooth muscle cells). In particular, the term refers to a Peripheral Vascular Disease (PVD). A “Peripheral Vascular Disease” or “PVD” refers to a disease/disorder/condition which can be treated and/or prevented using multipotent cells, a cell preparation or pharmaceutical composition of the invention. In particular, a Peripheral Vascular Disease includes diseases and circulation disorders of blood vessels outside the heart and brain and includes without limitation functional PVD, organic PVD, Peripheral Artery Disease (PAD), intermittent claudication, critical limb ischemia, artherosclerotic occlusive disease, arteriosclerosis, traumatic injury of vessels and inflammatory arteritides. A PVD can be characterized by a functional blockage of blood vessels. By way of example, in PAD, a condition similar to coronary artery disease and carotid artery disease, fatty deposits build up along artery walls and affect blood circulation, mainly in arteries leading to the legs and feet. Patients with PAD have a higher risk of stroke and heart attack due to the risk of blood clots.

In aspects of the invention the PVD is PAD. In other aspects the PVD is associated with diabetes. In further aspects, the cells, cell preparations and compositions of the invention are used to treat foot ulcers and other ischemic tissues refractory to traditional therapies.

In aspects of the invention, the disease is critical limb ischemia. In other aspects of the invention, the disease is intermittent claudication. Intermittent claudication is an ischemic disease of skeletal muscle characterized by repeated bouts of ischemia-reperfusion. Symptoms of the disease include pain, aching or fatigue that occurs in a muscle with an inadequate blood supply that is stressed by exercise.

In further aspects of the invention, the disease is a skeletal muscle injury caused by ischemia and/or reperfusion.

The multipotent cells, preparations, compositions, cells and methods of the invention may also have application in the treatment of coronary diseases. A coronary disease is a disease/disorder of cardiac function due to an imbalance between myocardial function and the capacity of coronary vessels to supply sufficient blood flow for normal function. Examples of coronary diseases/disorders associated with coronary disease which may be treated with the cells, preparations, compositions and methods described herein include myocardial ischemia, angina pectoris, coronary aneurysm, coronary thrombosis, coronary vasospasm, coronary artery disease, coronary heart disease, coronary occlusion and coronary stenosis.

### Preparation of Multipotent Cells

Multipotent cells may be produced by culturing an enriched hematopoietic cell preparation, preferably derived from umbilical cord blood, under proliferation conditions, in particular in the presence of or media comprising one or more positive growth factors and isolating the multipotent cells in the culture. In a particular aspect, the enriched hematopoietic cell preparation essentially comprises Lin^{neg} cells. An enriched hematopoietic cell preparation may be prepared using positive or negative selection techniques known in the art. For example, a source of hematopoietic cells (e.g., umbilical cord blood) can be treated to remove mature myeloid cells and lymphocytes using antibodies specific to the mature cells (e.g., CD2, CD3, CD14, CD16, CD19, CD24, CD56, CD66b, glycophorin A and/or dextran). A source of hematopoietic cells generally contains a minimum total nucleated cell count of about 50-1000 million cells, 500-1000 million cells, 500 to 700 million cells, 600 to 700 million cells, in particular 650 million cells, to ensure a sufficient cell dose in the final multipotent cell preparation.

Proliferation conditions are those conditions that give rise to multipotent cells. The proliferation conditions preferably involve culturing the enriched hematopoietic cell preparation in the presence of or media comprising one or more positive growth factors for a sufficient time, in particular a sufficient time to enable the cells to complete sufficient cell cycles, to form multipotent cells. Positive growth factors are growth factors that promote and maintain cell proliferation.

A positive growth factor may be human in origin, or may be derived from other mammalian species when active on human cells. The following are representative examples of positive growth factors which may be employed to produce multipotent cells: all members of the fibroblast growth factor (FGF) family including FGF-4 and FGF-2, epidermal growth factor (EGF), stem cell factor (SCF), thrombopoietin (TPO), Flt-3 ligand, interleukin-3 (II-3), interleukin-6 (IL-6), neural growth factor (NGF), VEGF, Granulocyte-Macrophage Growth Factor (GM-CSF), HGF, Hox family, and Notch.

Preferably the positive growth factors or combination of growth factors used to produce the multipotent cells are fibroblast growth factor (FGF) (e.g. FGF-4 and FGF-2), IL-3, stem cell factor (SCF), Flt-3 ligand, thrombopoietin (TPO), granulocyte macrophage-colony stimulating factor (GM-CSF), and neural growth factor (NGF). In embodiments of the invention, FGF (e.g. FGF-4 or FGF-2) is used with SCF and Flt-3 ligand; FGF is used with TPO; or TPO is used with SCF and FLT3ligand.

In an aspect of the invention the proliferation conditions involve using FGF-4 or FGF-2, SCF and Flt3-ligand, in particular FGF-4, SCF and Flt3-ligand, to prepare multipotent cells. In another aspect the proliferation conditions involve using TPO, SCF and Flt-3 ligand to prepare multipotent cells. In another aspect the proliferation conditions involve using NGF, SCF, and Flt-3 to prepare multipotent cells.

The growth factors may be used in combination with equal molar or greater amounts of a glycosaminoglycan such as heparin sulfate.

Growth factors may be commercially available or can be produced by recombinant DNA techniques and purified to various degrees. For example, growth factors are commercially available from several vendors such as, for example, Genzyme (Framingham, Mass.), Genentech (South San Francisco, Calif.), Amgen (Thousand Oaks, Calif.), R&D Systems (Minneapolis, Minn.) and Immunex (Seattle, Wash.). Some growth factors may be purified from culture media of cell lines by standard biochemical techniques. Thus, it is intended that molecules having similar biological activity as wild-type or purified growth factors (e.g., recombinantly produced or mutants thereof) are intended to be used within the spirit and scope of the invention.

An effective amount of a positive growth factor is used in the culture medium. Generally, the concentration of a positive growth factor in the culture medium is between 10 and 150 ng/ml, preferably 20 to 100 ng/ml or 25 to 100 ng/ml, more preferably 20 to 50 ng/m, 20 to 60 ng/ml, 20 to 55 ng/ml, 25 to 55 ng/ml, most preferably 25 to 50 ng/ml. The growth factors are typically applied at sufficient intervals to maintain high proliferation levels. In an embodiment, the growth factors are applied about 2-4 times per week, preferably 2-3 times per week.

The culture medium may comprise conditioned medium, non-conditioned medium, or embryonic stem cell medium. Examples of suitable conditioned medium include IMDM, DMEM, or αMEM, conditioned with embryonic fibroblast cells (e.g. human embryonic fibroblast cells or mouse embryonic fibroblast cells), or equivalent medium. Examples of suitable non-conditioned medium include Iscove's Modified Delbecco's Medium (IMDM), DMEM, or αMEM, RPMI, StemSpan, or equivalent medium. The culture medium may comprise serum (e.g. bovine serum, fetal bovine serum, calf bovine serum, horse serum, human serum, or an artificial serum substitute [e.g. 1% bovine serum albumin, 10 μg/ml bovine pancreatic insulin, 200 μg/ml human transferrin, 10⁻⁴M β-mercaptoethanol, 2 mM L-glutamine and 40 μg/ml LDL (Low Density Lipoproteins)], or it may be serum free.

In an embodiment, the culture medium is serum free to provide multipotent cells that are free of serum proteins or biomolecules that may bind to the surface of the cells.

In a particular embodiment, the culture medium comprises FGF-4, SCF and Flt-3 ligand in a serum free medium, in particular BIT or STI (sometimes referred to herein as “FSF1 medium”). The concentration of FGF-4, SCF and Flt-3 ligand in the culture medium can be between about 10 to 75 ng/ml, 15 to 60 ng/ml, 20 to 60 ng/ml, 30 to 60 ng/ml, 20-55 ng/ml, 25-55 ng/ml, 25-50 ng/ml, 40 to 55 ng/ml, 45 to 55 ng/ml, or 45 to 50 ng/ml preferably 25-50 ng/ml.

The enriched hematopoietic cell preparation may be seeded into the culture medium at a concentration of about 1×10³ cells/ml to 5×10⁷ cells/ml, 1×10⁴ cell/ml to 1×10⁵ cells/ml, or 1×10⁴ cells/ml to 5×10⁴ cells/ml.

The proliferation conditions entail culturing the enriched hematopoietic cell preparation for a sufficient period of time to produce multipotent cells. The enriched hematopoietic cells are generally maintained so that the cells complete about 1-100 cell cycles, preferably 5-75 cell cycles, more preferably 2-50, 2-40 or 2-20, most preferably at least about 2-10 or 4-5 cell cycles. The enriched hematopoietic cells are typically maintained in culture for about 4 to 40 days, preferably about 2-20 days, more preferably at least or about 2-15 days, 2-12 days, 4-10 days, or 8-12 days, and most preferably at least about 4-8 days, 8-12 days, 8-10 days or 8 days.

The frequency of feeding hematopoietic cells is selected to promote the survival and growth of multipotent cells. In embodiments, the hematopoietic cells are fed once, twice, three times or four times a week. The cells may be fed by replacing the entirety of the culture media with new media.

The cells in culture may be selected for hematopoietic stem and progenitor cells (e.g. CD45⁺HLA-ABC⁺ cells) at a frequency to promote the survival and growth of multipotent cells. In aspects of the invention, cells enriched for hematopoietic stem and progenitor cells (e.g. CD45⁺HLA-ABC⁺ cells) are reselected at intervals, preferably weekly, through positive or negative selection techniques known in the art.

Multipotent cells may be produced on a large-scale, for example multipotent cells may be isolated and/or expanded in bioreactors.

In an aspect of the invention, the multipotent cells are characterized by one or more of the following:
(a) CD45⁺;
(b) HLA-ABC⁺;
(c) having characteristic of or capable of forming EPCs;
(d) capable of differentiating or ability to differentiate into endothelial cells;
(e) having characteristics of or capable of forming or ability to form pericytes;
(f) capable of differentiating or ability to differentiate into muscle cells (in particular smooth muscle cells);
(g) stem cell factor receptor (KIT)+;
(h) FLT3ligand receptor+;
(i) FGF receptor+;
(j) express embryonic stem cell proteins such as Oct4, Stage Specific Embryonic Antigen-3 (SSEA3), nanog, and/or Stage Specific Embryonic Antigen-4 (SSEA4);
(k) Flk-1⁺;
(l) CD34⁺;
(m) CD38⁺; and
(n) derived from umbilical cord blood.

Multipotent cells may comprise cells with the characteristics (a) and (c); (a), (b), and (c); (a), (b) and (e); (a), (b), (c) and (d); (a), (b), (c), (d) and (e); (a), (b), (c) and (k); (a), (b), (c), (d), (e), (f), and (g); (a) through (e) inclusive; (a) through (f) inclusive; (a) through (g) inclusive; (a) through (h) inclusive; (a) through (i) inclusive; (a) through (j) inclusive; (a) through (k) inclusive; (a) through (l) inclusive; (a) through (j) inclusive, and (l); (a) through (i) inclusive and (k); or (a) through (n) inclusive.

In aspects of the invention the multipotent cells are CD45⁺HLA-ABC⁺Lin⁻. In aspects of the invention, the multipotent cells have the phenotypic characteristics of the post-culture cells in Table 2. In aspects of the invention the multipotent cells have characteristics associated with EPC's (e.g, Flk1+). In aspects of the invention the multipotent cells have characteristics associated with pericytes (e.g. desmin+).

Multipotent cells may be expanded using proliferation conditions described herein or known in the art (e.g., using one or more positive growth factors).

In aspects of the invention a multipotent cell preparation comprises at least 60%, 70%, 80% or 85% CD45+ cells. In aspects of the invention, a multipotent cell preparation comprises about 1-5×10⁷ cells, preferably 2×10⁷ cells.

Production of ECs, Pericyte and/or Muscle Cell Preparations

The multipotent cells may be induced to differentiate into EPCs, endothelial cells, pericytes, muscle cells (in particular smooth muscle cells), or vascular or muscle tissues in vitro or in vivo. In an aspect, the multipotent cells can be induced to differentiate into endothelial cells, in particular cells that exhibit morphological, physiological, functional, and/or immunological features of endothelial cells. In another aspect, the multipotent cells can be induced to differentiate into pericytes, in particular cells that exhibit morphological, physiological, functional, and/or immunological features of pericytes. In another aspect, the multipotent cells can be induced to differentiate to muscle cells, in particular smooth muscle cells, that exhibit morphological, physiological, functional, and/or immunological features of muscle cells.

Endothelial cells obtained by a method of the invention can be characterized by one or more of the following properties:
(a) CD31⁺;
(b) CD 133⁺;
(c) express VE-cadherin;
(d) express von Willebrand factor;
(e) express CD34;
(f) Flk-1⁺;
(g) elongated cells;
(h) ability to grow into a network of vessel-like structures in vitro and in vivo; and;
(i) ability to secrete growth factors; and
(j) capable of contributing to or ability to contribute to vessel formation in vitro and in vivo.

EPCs obtained by a method of the invention can be characterized by expression of Flk-1 and ability to differentiate into endothelial cells.

Pericytes obtained by a method of the invention can be characterized by one or more of the following properties:
(a) express CD31, NG2 chondroitin sulphate proteoglycan, desmin, antiopoietin-1, osteonectin, and/or Thy-1; and
(b) capable of contributing to or ability to contribute to vessel formation in vitro and in vivo

Muscle cells obtained by a method of the invention can be characterized by one or more of the following properties:
(a) express MyoD;
(b) express muscle actin;
(c) express myosin heavy chain;
(d) express Ang-1 and/or PDGF-13; and
(e) capable of contributing or ability to contribute to vessel formation in vitro and in vivo

In aspects of the invention, a cell population of the invention essentially comprises or comprises at least about 60%, 70%, 80%, 90%, 95%, or 98% EPCs, endothelial cells, pericytes, and/or muscle cells (in particular smooth muscle cells), such cells identified as being positive for one, two, or three of any of the phenotypic markers disclosed herein.

In an aspect of the invention, a purified cell preparation is provided comprising essentially or consisting essentially of EPCs, endothelial cells, pericytes and/or muscle cells (in particular smooth muscle cells), in particular at least about 50%, 60%, 70%, 80%, 90%, 95%, or 99%, preferably at least 80% or 90% EPCs, endothelial cells, pericytes and/or muscle cells (in particular smooth muscle cells), wherein the EPCs express Flk-1, the endothelial cells express CD31 and/or CD133, the pericytes express CD31, NG2 chondroitin sulphate proteoglycan, desmin, angiopoietin-1, osteonectin, and/or Thy-1, and the muscle cells express MyoD, muscle actin, and/or myosin heavy chain.

Markers can be detected using any suitable immunological technique such as flow immunocytochemistry for cell-surface markers or immunohistochemistry of, for example, fixed cells or tissues for intracellular or cell-surface markers. A cell is positive for a marker if it shows substantially higher staining using specific antibody in an immunocytochemistry, flow cytometry assay or immunohistochemistry technique compared with a control. Tissue-specific gene products can be detected at the mRNA level by Northern blot analysis, dot-blot hybridization analysis, or by reverse transcriptase initiated polymerase chain reaction (RT-PCR) using sequence-specific primers. Sequence information for markers may be obtained from public databases such as GenBank.

Cell preparations of the invention can be characterized by morphological features of precursor or mature endothelial cells or muscle cells. For example, the ECs can be elongated and adherent cells. EPCs, endothelial cells, pericytes and muscle cells of preparations of the invention can also be characterized by functional criteria. For example, ECs may be assessed for their ability to form capillaries in 3-D cultures or contribute to vessel formation. The ability of the ECs to contribute to vessel formation in vivo can be demonstrated using suitable animal models such as the animal models disclosed in the Examples.

EPCs, endothelial cells, pericytes and muscle cells (in particular smooth muscle cells) can be obtained by culturing multipotent cells in a special growth environment that enriches and/or expands cells with the desired phenotype. The growth environment may specifically direct differentiation into ECs (particularly endothelial cells), pericytes or muscle cells (in particular smooth muscle cells), promote outgrowth of the desired cells, inhibit growth of other cell types or perform any combination of these activities. Examples of culture media to produce muscle cells (in particular smooth muscle cells) include muscle specific cell culture media available from Celprogen and StemCell Technologies. An example of culture medium which can be used to produce endothelial cells includes M119 medium with serum (10%), supplemented with endothelial growth factor supplement.

In an aspect, the invention provides a method for producing an isolated and purified cell preparation comprising endothelial cells disclosed herein comprising culturing multipotent cells previously grown in culture medium comprising FGF-4, SCF, and Flt-3 ligand, under suitable differentiation conditions to induce the mulitpotent cells to endothelial cells. Endothelial cells may be obtained by growing multipotent cells on media that induces differentiation of the cells to endothelial cells (e.g. medium supplemented with differentiation factors such as EGF or VEGF). Endothelial cells may be identified based on expression of endothelial specific markers such as CD31.

In another aspect, the invention provides a method for producing an isolated and purified cell preparation comprising pericytes disclosed herein comprising culturing multipotent cells previously grown in culture medium comprising FGF-4, SCF, and FLT-3 ligand, under suitable culture conditions or differentiation conditions to induce mulitpotent cells to pericytes. Pericytes may be identified based on expression of specific markers such as CD31, NG2 chondroitin sulphate proteoglycan, desmin, angiopoietin-1, osteonectin, and/or Thy-1.

In another aspect, the invention provides a method for producing an isolated and purified cell preparation comprising muscle cells, in particular smooth muscle cells, disclosed herein comprising culturing multipotent cells previously grown in culture medium comprising FGF-4, SCF, and FLT-3 ligand, under suitable differentiation conditions to induce mulitpotent cells to muscle cells, in particular smooth muscle cells. Muscle cells, in particular smooth muscle cells, may be identified based on expression of specific markers such as myosin heavy chain, MyoD, muscle actin.

After differentiation of the multipotent cells into EPCs, endothelial cells, pericytes or muscle cells (in particular smooth muscle cells) disclosed herein, the cells may be separated to obtain a population of cells largely or essentially consisting of the EPCs, endothelial cells, pericytes or muscle cells. This may be accomplished using various separation procedures such as antibody or lectin mediated adherence or sorting for cell surface markers. In aspects of the invention, positive selection of the cells may be carried out using antibodies to identify tissue specific cell surface markers or negative selection may be carried out using cell specific markers (e.g., CD31, myoD, muscle actin, and/or myosin heavy chain).

Cells in the cell preparations of the invention can be used to prepare a cDNA library relatively uncontaminated with cDNA preferentially expressed in cells from other lineages, and they can be used to prepare antibodies that are specific for particular markers of EPCs, endothelial cells, pericytes or muscle cells (in particular smooth muscle cells).

Prior to use of a cell preparation of the invention, the number of EPCs, endothelial cells, pericytes or muscle cells (in particular smooth muscle cells) in the preparation can be increased by causing them to proliferate further in culture. This can be accomplished by culturing the cells in the presence of or in media comprising one or more positive growth factors. For example, positive growth factors which can be used for proliferation of the cells are fibroblast growth factors (e.g., FGF-2 and FGF-4), epidermal growth factor (EGF), functional homologs, and other factors that bind the EGF receptor; platelet-derived growth factor (PDGF), and insulin-like growth factor (IGF). It may be beneficial to include differentiation factors in the medium to maintain preferential growth of EPCs, endothelial cells, pericytes or muscle cells (in particular smooth muscle cells). Expansion of the number of EPCs, endothelial cells, pericytes or muscle cells allows large populations of EPCs, endothelial cells, pericytes and muscle cells (in particular smooth muscle cells) to be produced.

### Modification of Cells

A cell preparation or pharmaceutical composition of the invention may be derived from or comprised of cells that have been genetically modified (transduced or transfected) either in nature or by genetic engineering techniques in vivo or in vitro.

Cells in cell preparations and compositions of the invention can be modified by introducing mutations into genes in the cells (or the cells from which they are obtained) or by introducing transgenes into the cells. Insertion or deletion mutations may be introduced in a cell using standard techniques. A transgene may be introduced into cells via conventional techniques such as calcium phosphate or calcium chloride co-precipitation, DEAE-dextran-mediated transfection, lipofection, electroporation, or microinjection. Suitable methods for transforming and transfecting cells can be found in Sambrook et al. [Sambrook, Fritsch, & Maniatis, Molecular Cloning: A Laboratory Manual, Second Edition (1989) Cold Spring Harbor Laboratory Press, Cold Spring Harbor, N.Y], and other laboratory textbooks. By way of example, a transgene may be introduced into cells using an appropriate expression vector including but not limited to cosmids, plasmids, or modified viruses (e.g. replication defective retroviruses, adenoviruses and adeno-associated viruses). Transfection is easily and efficiently obtained using standard methods including culturing the cells on a monolayer of virus-producing cells (see Van der Putten, 1985, Proc Natl Acad Sci USA.; 82:6148-52; Stewart et al. 1987, EMBO J. 6:383-388).

A gene encoding a selectable marker may be integrated into cells of a cell preparation or composition of the invention. For example, a gene which encodes a protein such as β-galactosidase, chloramphenicol acetyltransferase, firefly luciferase, or a fluorescent protein marker may be integrated into the cells. Examples of fluorescent protein markers are the Green Fluorescent Protein (GFP) from the jellyfish A. victoria, or a variant thereof that retains its fluorescent properties when expressed in vertebrate cells. (For example, the GFP variants described in Heim et al, 1994, Proc. Natl. Acad. Sci. 91:12501; M. Zernicka-Goetz et al, 1997, Development 124:1133-1137; Okabe, M. et al, FEBS Letters 407:313-319, 1997; and EGFP commercially available from Clontech Palo Alto, Calif.).

Another aspect of the present invention relates to genetically engineering the cells in the cell preparations and compositions of the invention in such a manner that they or cells derived therefrom produce, in vitro or in vivo, polypeptides, hormones and proteins to not normally produced in the cells in biologically significant amounts, or produced in small amounts but in situations in which regulatory expression would lead to a therapeutic benefit. For example, the cells could be modified such that a protein normally expressed will be expressed at much lower levels. These products would then be secreted into the surrounding media or purified from the cells. The cells formed in this way can serve as continuous short term or long term production systems of the expressed substance.

Thus, genes can be introduced into cells which are then injected into a recipient where the expression of the gene will have a therapeutic effect. The technology may also be used to produce additional copies of essential genes to allow augmented expression by ECs, pericytes and muscle cells (in particular smooth muscle cells) of certain gene products in vivo. These genes can be, for example, cell membrane proteins, cytokines, or adhesion molecules, or “rebuilding” proteins important in tissue repair.

By way of example, ECs of the invention may genetically engineered so that they produce an angiogenic growth factor such as VEGF, a fibroblast growth factor such as basic FGF or FGF-4, placental growth factor, hepatocyte growth factor, angiogenin, angiopoietin-1, pleiotrophin, transforming growth factor (alpha. or beta.), or tumor necrosis factor alpha. ECs produced by methods of the invention can also produce a natiuretic peptide such as an atrial natiuretic peptide (ANP) or a brain natriuretic peptide (BNP), prostacyclin synthase, nitric oxide synthase, angiostatin, endostatin, erythropoietin (EPO), GM-CSF, or an interleukin such as IL-1, 2, 3, 4, 5, 6, 7, 8, 9, 10, 11, 12, 13, 14, 15, 16, 17, or 18. ECs of the invention can also be engineered to produce an adhesion molecule such as a selectin (e.g., E, L, or P selectin), an extracellular matrix protein (e.g., collagen type I, III, or IV; fibronectin; laminin; or vitronectin), an integrin (e.g., α₅β₁), or an intracellular adhesion molecule such as ICAM or a vascular cell adhesion molecule (VCAM).

Multipotent cells used to produce cell preparations can also be modified with genetic material of interest. The modified cells can be cultured in vitro under suitable conditions as disclosed herein so that they differentiate into EPCs, endothelial cells, pericytes or muscle cells (in particular smooth muscle cells). The EPCs, endothelial cells, pericytes or muscle cells (in particular smooth muscle cells) are able to express the product of the gene expression or secrete the expression product. These modified cells can be administered to a target tissue where the expressed product will have a beneficial effect. In a further embodiment, the transduced multipotent cells can be induced in vivo to differentiate into EPCs, endothelial cells, pericytes or muscle cells (in particular smooth muscle cells) that will express the gene product. For example, the transduced multipotent cells may be administered to induce production of EPCs, endothelial cells, pericytes or muscle cells (in particular smooth muscle cells) having the transduced gene. The cells may is be administered in admixture with each other or separately and may be delivered to a targeted area. The cells can be introduced intravenously and home to the targeted area. Alternatively, the cells may be used alone and caused to differentiate in vivo.

### Applications

The multipotent cells, cell preparations and compositions of the invention and cells obtained therefrom, can be used in a variety of methods (e.g. transplantation) and they have numerous uses in the field of medicine. They may be used for the replacement of cells, body tissues, organs, components or structures which are missing or damaged due to trauma, age, metabolic or toxic injury, disease, idiopathic loss, or any other cause.

Transplantation or grafting, as used herein, can include the steps of isolating multipotent cells or a cell preparation according to the invention and transferring the multipotent cells or cells in the preparation into a mammal or a patient. Transplantation can involve transferring the cells into a mammal or a patient by injection of a cell suspension into the mammal or patient, surgical implantation of a cell mass into a tissue or organ of the mammal or patient, or perfusion of a tissue or organ with a cell suspension. The route of transferring the cells may be determined by the requirement for the cells to reside in a particular tissue or organ and by the ability of the cells to find and be retained by the desired target tissue or organ. Where the transplanted cells are to reside in a particular location, they can be surgically placed into a tissue or organ or simply injected into the bloodstream if the cells have the capability to migrate to the desired target organ.

The invention may be used for autografting (cells from an individual are used in the same individual), allografting cells (cells from one individual are used in another individual) and xenografting (transplantation from one species to another). Thus, the multipotent cells, cell preparations and pharmaceutical compositions of the invention, and cells obtained therefrom, may be used in autologous or allogenic transplantation procedures to improve an EPC, endothelial cell, pericyte and/or muscle cell deficit.

In an aspect of the invention, the multipotent cells and/or newly created cell preparations and cells therefrom can be used in both cell therapies and gene therapies aimed at alleviating disorders and diseases involving EPCs, endothelial cells, pericytes and/or muscle cells (in particular smooth muscle cells). The invention obviates the need for human tissue to be used in various medical and research applications.

The cell therapy approach involves the use of transplantation of the multipotent cells and/or the newly created cell preparations comprising EPCs, endothelial cells, pericytes and/or muscle cells (in particular smooth muscle cells) as a treatment for a disease disclosed herein (e.g., a PVD). In an aspect, the steps in this application include: (a) producing multipotent cells or a cell preparation as described herein; and (b) allowing the cells to form functional connections either before or after a step involving transplantation of the cells or cell preparation. The gene therapy approach also involves multipotent cells and cell preparations, however, following the culturing step in proliferation conditions, the newly created cells are transfected with an appropriate vector containing a cDNA for a desired protein and the cells are optionally differentiated, followed by a step where the modified cells are transplanted.

In either a cell or gene therapy approach, therefore, multipotent cells or cell preparations of the invention or cells therefrom can be transplanted in, or grafted to, a patient in need. Thus, the multipotent cells and cell preparations or cells therefrom can be used to replace EPCs, endothelial cells, pericytes and/or muscle cells (in particular smooth muscle cells) in a patient in a cell therapy approach, useful in the treatment of a disease disclosed herein (e.g., a PVD). These cells can be also used as vehicles for the delivery of specific gene products to a patient.

The invention also provides a method of treating a patient with a disease disclosed herein, in particular a PVD, more particularly PAD, intermittent claudication, or critical limb ischemia, comprising transferring multipotent cells, a cell preparation or composition of the invention or cells therefrom into the patient. In aspects of the invention, the cells, preparation or composition are transferred by intramuscular or intravenous administration.

A method of the invention may involve producing or obtaining EPCs, endothelial cells, pericytes and/or muscle cells (in particular smooth muscle cells) for autologous transplantation from a patient's own hematopoietic cells comprising: (a) obtaining a sample comprising hematopoietic cells from the patient, preferably from fresh or cryopreserved umbilical cord blood; (b) separating out an enriched cell preparation comprising Lin^{neg} stem cells and progenitor cells; (c) culturing the cells under proliferation conditions to produce multipotent cells, preferably CD45⁺HLA-ABC⁺ cells; and (d) culturing the multipotent cells under suitable differentiation conditions to produce a cell preparation comprising EPCs, endothelial cells, pericytes and/or muscle cells (in particular smooth muscle cells); and (e) transferring the multipotent cells of (c) or a cell preparation of (d) to the patient. In an aspect, the multipotent cells comprise endothelial, smooth muscle and/or striated muscle precursor cells.

A method of the invention may involve producing or obtaining EPCs, endothelial cells, pericytes and/or muscle cells (in particular smooth muscle cells) for allogeneic transplantation comprising: (a) obtaining a sample comprising hematopoietic cells from a donor subject, preferably from fresh or cryopreserved umbilical cord blood; (b) separating out an enriched cell preparation comprising Lin^{neg} stem cells and progenitor cells; (c) culturing the cells under proliferation conditions to produce multipotent cells, preferably CD45⁺HLA-ABC⁺ cells; (d) culturing the multipotent cells under suitable differentiation conditions to produce a cell preparation comprising EPCs, endothelial cells, pericytes and/or muscle cells; and (e) transferring the multipotent cells of (c) or a cell preparation of (d) to another subject to treat a disease disclosed herein, in particular PVD. In an aspect, the multipotent cells comprise endothelial, smooth muscle and/or striated muscle precursor cells.

In an aspect of the invention, a method is providing for improving perfusion of ischemic tissue in a subject comprising: (a) obtaining a sample comprising hematopoietic cells from the patient, preferably from fresh or cryopreserved umbilical cord blood; (b) separating out an enriched cell preparation comprising Lin^{neg} stem cells and progenitor cells; (c) culturing the cells under proliferation conditions to produce multipotent cells, preferably CD45⁺HLA-ABC⁺ cells; and (d) culturing the multipotent cells under suitable differentiation conditions to produce a cell preparation comprising EPCs, endothelial cells, pericytes and/or muscle cells (in particular smooth muscle cells); and (e) transferring the multipotent cells of (c) or a cell preparation of (d) to the patient. In an aspect, the multipotent cells comprise endothelial, smooth muscle and/or striated muscle precursor cells.

In a particular aspect of the invention, a method is provided for treating a subject with critical limb ischemia comprising: (a) obtaining a sample comprising hematopoietic cells from the patient, preferably from fresh or cryopreserved umbilical cord blood or bone marrow; (b) separating out an enriched cell preparation comprising Lin^{neg} stem cells and progenitor cells; (c) culturing the cells under proliferation conditions to produce multipotent CD45⁺HLA-ABC⁺ cells comprising endothelial, smooth muscle and/or striated muscle precursor cells; and (d) transferring the multipotent cells to the subject.

The invention also contemplates a pharmaceutical composition comprising multipotent cells, a cell preparation of the invention or EPCs, endothelial cells, pericytes and/or muscle cells (in particular smooth muscle cells) therefrom and a pharmaceutically acceptable carrier, excipient, or diluent. The pharmaceutical compositions herein can be prepared by per se known methods for the preparation of pharmaceutically acceptable compositions which can be administered to subjects, such that an effective amount of the active substance is combined in a mixture with a pharmaceutically acceptable vehicle. Suitable vehicles are described, for example, in the standard texts Remington: The Science and Practice of Pharmacy (21^{5t} Edition. 2005, University of the Sciences in Philadelphia (Editor), Mack Publishing Company), and in The United States Pharmacopeia: The National Formulary (USP 24 NF19) published in 1999. On this basis, the compositions include, albeit not exclusively, solutions of the cell preparations or EPCs, endothelial cells, pericytes and/or muscle cells (in particular smooth muscle cells) therefrom in association with one or more pharmaceutically acceptable vehicles or diluents, and contained in buffered solutions with a suitable pH and iso-osmotic with the physiological fluids.

An implantable medical device (e.g., a stent, including a coated stent, graft such as a vascular graft, sheet, hollow tube, or valve) can include multipotent cells, ECs, pericytes, or muscle cells (in particular smooth muscle cells), cell preparations or compositions of the invention. For example, the ECs can be seeded onto a device. (See U.S. Patent Publication No. US-2002-0160033.) For example, ECs can be used to form living vascular grafts, including arterial, venous, and renal grafts or living prosthetic valves for venous and cardiac applications. ECs also can be used to create implantable sphincters or reline the aorta in patients with shaggy aorta.

The cells, preparations, compositions or treatment methods of the invention may be used with one or more other treatments or treatment methods effective for the same disease, in particular PVD. For example, the treatment methods of the invention may be used in combination with antiplatelet drugs, anticoagulants, cholesterol lowering drugs, calcium channel blockers, angioplasty, endarterectomy, grafting or bypass. The treatment methods of the invention may also be used with one or more immunosuppressive agents. A treatment or treatment method may be used prior to or at the same time as the patient receives a transplant of multipotent cells, a cell preparation or composition of the invention, or cells therefrom.

A cell preparation composition, medicament, or treatment of the invention may comprise a single unit dosage of multipotent cells, EPC's, endothelial cells, pericytes and/or muscle cells (in particular smooth muscle cells). A “unit dosage” refers to a unitary i.e. a single dose which is capable of being administered to a patient, and which may be readily handled and packed, remaining as a physically and chemically stable unit dose comprising either the cells, cell preparations or compositions as such or a mixture with one or more pharmaceutical excipients, carriers, or vehicles. A cell preparation, composition or unit dose may comprise a cell dose of greater than 1×10⁵ to 5×10⁸, 1×10⁶ to 1×10⁸, or 1×10⁷ to 5×10⁷, in particular greater than 2.0×10⁷ cells.

Still another aspect of the invention is a kit for producing cell preparations of the invention comprising multipotent cells capable of differentiating into EPCs, endothelial cells), pericytes, or muscle cells (in particular smooth muscle cells) both in vitro and in vivo. The kit includes the reagents for a method of the present invention for producing a cell preparation comprising ECs (particularly endothelial cells), pericytes and/or muscle cells (in particular smooth muscle cells). This kit preferably would include at least one differentiation factor, and instructions for use. Further the invention contemplates a kit comprising multipotent cells, a cell preparation or composition of the invention or cells therefrom in kit form. A kit may comprise a package which houses a container which contains multipotent cells, a preparation or composition of the invention and also houses instructions for administering the preparation or composition to a subject. Associated with such container can be various written materials such as a notice in the form prescribed by a governmental agency regulating the labeling, manufacture, use or sale of pharmaceuticals or biological products, which notice reflects approval by the agency of manufacture, use, or sale for human administration. A kit can also comprise cell preparations of the invention or cells therefrom for conducting the screening and testing methods disclosed herein.

In an aspect, cell preparations and pharmaceutical compositions disclosed herein can be used for toxicity testing for drug development testing. Toxicity testing may be conducted by culturing the cell preparations or pharmaceutical compositions or cells obtained or derived therefrom in a suitable medium and introducing a substance, such as a pharmaceutical or chemical, to the culture. The cells are examined to determine if the substance has had an adverse effect on the culture. Drug development testing may be done by developing derivative cell lines which may be used to test the efficacy of new drugs. Affinity assays for new drugs may also be developed from the cell preparations or cell lines. Using a method of the invention it is possible to identify substances, in particular drugs, that are potentially toxic to EPCs, endothelial cells, pericytes and/or muscle cells (in particular smooth muscle cells).

The cell preparations of the invention may be used to screen for potential therapeutics that modulate development or activity of EPCs, endothelial cells, pericytes and/or muscle cells (in particular smooth muscle cells). In particular, the EPCs, endothelial cells, pericytes and/or muscle cells (in particular smooth muscle cells) of a cell preparation of the invention may be subjected to a test substance, and the effect of the test substance may be compared to a control (e.g. in the absence of the substance) to determine if the test substance modulates development or activity of the cells.

In an aspect of the invention a method is provided for using cell preparations of the invention to assay the activity of a test substance comprising the steps of:
a) culturing multipotent cells (e.g., CD45⁺HLA-ABC⁺) in vitro under suitable differentiation conditions to induce production of EPCs, endothelial cells, pericytes and/or muscle cells (in particular smooth muscle cells);
b) exposing the cultured cells in step (a) to a test substance; and
c) detecting the presence or absence of an effect of the test substance on the survival of the EPCs, endothelial cells, pericytes and/or muscle cells or on a morphological, functional, or physiological characteristic and/or molecular biological property of such cells, whereby an effect altering cell survival, a morphological, functional, or physiological characteristic and/or a molecular biological property of the cells indicates the activity of the test substance.

In another aspect a method is provided for using cell preparations of the invention to screen a potential new drug to treat a disease or disorder involving EPCs, endothelial cells, pericytes and/or muscle cells (in particular smooth muscle cells) comprising the steps of:
(a) obtaining hematopoietic cells from a sample from a patient with a disease or disorder disclosed herein, in particular a PVD;
(b) preparing from the hematopoietic cells an enriched hematopoietic cell preparation comprising hematopoietic stem cells and progenitor cells (e.g., Lin^{neg} cells);
(c) culturing the enriched hematopoietic cell preparation under proliferation conditions to obtain multipotent cells (e.g., CD45⁺HLA-ABC⁺ cells);
(d) culturing the multipotent cells under suitable culture or differentiation conditions to produce EPCs, endothelial cells, pericytes and/or muscle cells;
(e) exposing the cultured cells in (c) or (d) to a potential new drug; and
(f) detecting the presence or absence of an effect of the potential new drug on the survival of the EPCs, endothelial cells, pericytes and/or muscle cells or on a morphological, functional, or physiological characteristic and/or molecular biological property of said cells, whereby an effect altering cell survival, a morphological, functional, or physiological characteristic and/or a molecular biological property of the cells indicates the activity of the potential new drug.

The invention also relates to the use of cell preparations and pharmaceutical compositions of the invention in drug discovery. The invention provides methods for drug development using the cell preparations and pharmaceutical compositions of the invention. The cell preparations and pharmaceutical compositions of the invention may comprise EPCs, endothelial cells, pericytes and/or muscle cells (in particular smooth muscle cells) that secrete novel or known biological molecules or components. In particular, culturing in the absence of serum may provide cells that have minimal interference from serum molecules and thus, may be more physiologically and topologically accurate. Therefore, proteins secreted by EPCs, endothelial cells, pericytes and/or muscle cells (in particular smooth muscle cells) described herein may be used as targets for drug development. Drugs can also be made to target specific proteins on EPCs, endothelial cells, pericytes and/or muscle cells described herein. In addition, drugs specific for regulatory proteins of EPCs, endothelial cells, pericytes and/or muscle cells may be used to arrest growth of cells. Any of the proteins can be used as targets to develop antibody, protein, antisense, aptamer, ribozymes, or small molecule drugs.

Agents, test substances, or drugs identified in accordance with a method of the invention or used in a method of the invention include but are not limited to proteins, peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], nucleic acids, ribozymes, carbohydrates, and small organic or inorganic molecules. An agent, substance or drug may be an endogenous physiological compound or it may be a natural or synthetic compound.

The cell preparations and pharmaceutical compositions of the invention can be used in various bioassays. In an embodiment, the cell preparations are used to determine which biological factors are required for proliferation or differentiation of EPCs, endothelial cells, pericytes and/or muscle cells (in particular smooth muscle cells). By using multipotent cells or cell preparations in a stepwise fashion in combination with different biological compounds (such as hormones, specific growth factors, etc.), one or more specific biological compounds can be found to induce differentiation of EPCs, endothelial cells, pericytes and/or muscle cells (in particular smooth muscle cells), or proliferation of EPCs, endothelial cells, pericytes and/or muscle cells (in particular smooth muscle cells). Other uses in a bioassay for the cells are differential display (i.e. mRNA differential display) and protein-protein interactions using secreted proteins from the cells. Protein-protein interactions can be determined with techniques such as a yeast two-hybrid system. Proteins from cell preparations and pharmaceutical compositions of the invention can be used to identify other unknown proteins or other cell types that interact with the cells. These unknown proteins may be one or more of the following: growth factors, hormones, enzymes, transcription factors, and translational factors. Bioassays involving cell preparations and pharmaceutical compositions of the invention, and the protein-protein interactions these cells form and the effects of protein-protein or cell-cell contact may be used to determine how surrounding tissue contributes to proliferation or differentiation of EPCs, endothelial cells, pericytes and/or muscle cells (in particular smooth muscle cells).

In an aspect of the invention cell preparations comprising, produced or derived from multipotent cells obtained after culturing a preparation from cord blood stem cells may be used to treat PVD. They may also be used in the treatment of genetic defects that result in nonfunctional EPCs, endothelial cells, pericytes and/or muscle cells (in particular smooth muscle cells). EPCs, endothelial cells, pericytes and/or muscle cells (in particular smooth muscle cells) obtained from multipotent cells derived from umbilical cord blood may be used for treating a disease disclosed herein, in particular a PVD, more particularly PAD, intermittent claudication or critical limb ischemia.

EPCs, endothelial cells, pericytes and/or muscle cells (in particular smooth muscle cells) generated in accordance with a method of the invention may be transfected with a vector that can express a desired protein such as a growth factor or growth factor receptor. These transfected cells may be transplanted into regions of vascular damage.

The multipotent cells, cell preparations, pharmaceutical compositions and EPCs, endothelial cells, pericytes and/or muscle cells (in particular smooth muscle cells) isolated or derived therefrom may be used as immunogens that are administered to a heterologous recipient. Administration of EPCs, endothelial cells, pericytes and/or muscle cells (in particular smooth muscle cells) obtained in accordance with the invention may be accomplished by various methods. Methods of administering EPCs, endothelial cells, pericytes and/or muscle cells (in particular smooth muscle cells) as immunogens to a heterologous recipient include without limitation immunization, administration to a membrane by direct contact (e.g. by swabbing or scratch apparatus), administration to mucous membranes (e.g. by aerosol), and oral administration. Immunization may be passive or active and may occur via different routes including intraperitoneal injection, intradermal injection, and local injection. The route and schedule of immunization are in accordance with generally established conventional methods for antibody stimulation and production. Mammalian subjects, particularly mice, and antibody producing cells therefrom may be manipulated to serve as the basis for production of mammalian hybridoma cell lines.

The cell preparations and compositions of the invention may be used to prepare model systems of disease. The cell preparations and compositions of the invention can also be used to produce growth factors, hormones, etc. In an aspect the invention provides a culture system from which genes, proteins, and other metabolites involved in proliferation or differentiation of ECs, pericytes and/or muscle cells (in particular smooth muscle cells) can be identified and isolated. The cells in a culture system of the invention may be compared with other cells (e.g. mature cells) to determine the mechanisms and compounds that stimulate production of ECs, pericytes and/or muscle cells (in particular smooth muscle cells).

The cell preparations of the invention can be used to screen for genes expressed in or essential for differentiation of ECs, pericytes and/or muscle cells (in particular smooth muscle cells). Screening methods that can be used include Representational Difference Analysis (RDA) or gene trapping with for example SA-lacZ (D. P. Hill and W. Wurst, 1993, Methods in Enzymology, 225: 664). Gene trapping can be used to induce dominant mutations (e.g. by deleting particular domains of the gene product) that affect differentiation or activity of ECs, pericytes and/or muscle cells (in particular smooth muscle cells) and allow the identification of genes expressed in or essential for differentiation of these cells.

The invention also relates to a method for conducting a regenerative medicine business, comprising: (a) a service for accepting and logging in samples from a client comprising hematopoietic cells capable of forming multipotent cells; (b) a system for culturing cells dissociated from the samples, which system provides conditions for producing multipotent cells and cell preparations comprising EPCs, endothelial cells, pericytes and/or muscle cells (in particular smooth muscle cells) therefrom; and/or (c) a cell preservation system for preserving multipotent cells and cell preparations generated by the system in (b) for later retrieval on behalf of the client or a third party. The method may further comprise a billing system for billing the client or a medical insurance provider thereof.

The invention features a method for conducting a cell business comprising identifying agents which influence the proliferation, differentiation, or survival of EPCs, endothelial cells, pericytes and/or muscle cells (in particular smooth muscle cells). Examples of such agents are small molecules, antibodies, and extracellular proteins. Identified agents can be profiled and assessed for safety and efficacy in animals. In another aspect, the invention contemplates methods for influencing the proliferation, differentiation, or survival of ECs, pericytes and/or muscle cells (in particular smooth muscle cells) by contacting the cells with an agent or agents identified by the foregoing method. The identified agents can be formulated as a pharmaceutical preparation, and manufactured, marketed, and distributed for sale.

In an embodiment, the invention provides a method for conducting a cell business comprising (a) identifying one or more agents which affect the proliferation, differentiation, function, or survival of ECs, pericytes and/or muscle cells (in particular smooth muscle cells) from a cell preparation of the invention; (b) conducting therapeutic profiling of agents identified in (a); or analogs thereof for efficacy and toxicity in animals; and (c) formulating a pharmaceutical composition including one or more agents identified in (b) as having an acceptable therapeutic profile. The method may further comprise the step of establishing a distribution system for distributing the pharmaceutical preparation for sale. The method may also comprise establishing a sales group for marketing the pharmaceutical preparation.

The invention also contemplates a method for conducting a drug discovery business comprising identifying factors that influence the proliferation, differentiation, function, or survival of EPCs, endothelial cells, pericytes and/or muscle cells (in particular smooth muscle cells) from cell preparations of the invention, and licensing the rights for further development.

The therapeutic efficacy of the cell preparations and agents identified using the methods of the invention can be confirmed in animal disease models. For example, the therapeutic efficacy of multipotent cells, a cell preparation or composition of the invention or cells obtained therefrom can be tested in PVD models including without limitation C57/BL6 mice that have undergone femoral artery ligation (Greve J M et al, J Magn Reson Imaging. 2006 November; 24(5):1124-32); a model of peripheral arterial disease (PAD) in rat skeletal muscle (Brown M D et al, Microcirculation. 2005 June; 12(4):373-81); a rat model of hind limb ischemia (Iwase T et al., Cardiovasc Res. 2005 Jun. 1; 66(3):543-51); mouse models of hind limb ischemia [Couffinhal, T. et al, Am J Pathol 152, 1667-79 (1998), Murohara, T. et al., J Clin Invest 101, 2567-78 (1998); Pesce, M. et al., Circ Res 93, e51-62 (2003)]; rat models of PVD induced by lauric acid or ergotamine plus epinephrine (Ogawa T et al., Vascul Pharmacol. 2004 February; 41(1):7-13); and the animal models disclosed herein.

Having now described the invention, the same will be more readily understood through reference to the following examples which are provided by way of illustration, and are not intended to be limiting of the present invention.

### Example 1

Umbilical cord blood cells and bone marrow cells will be used in this study. In some cases, bone marrow offers the advantage of using the patient's own cells (an HLA-match) for therapy. However, in some cases, such as with diabetic patients, endothelial precursor cells (EPCs) may not be suitable because of their reduced ability to contribute to revascularization, or harvesting bone marrow may be too stressful for a patient with advanced PVD and associated cardiac disease. The use of donor bone marrow from a related healthy donor may be acceptable. UCB storage banks are being set up worldwide and the ability of finding a matched donor is becoming more commonplace. Due to their accessibility UCB cells are well suited for cell therapy. Human bone marrow and umbilical cord blood will be tested for the ability to contribute EPCs and pericytes to the repair of ischemic tissue in a PVD model generated in NOD/SCID mice. A method will be utilized that expands the number of available progenitor cells through in vitro cell culture and cell selection during the culture period. These expanded populations of candidate cells will be tested in an ischemic mouse model for their ability to contribute to blood vessel formation and increased blood flow.

In vitro expansion of EPC from UCB and BM samples. Unfractionated samples and sub-populations of cells from human UCB or BM will be tested for their endothelial and muscle progenitor cell content, followed by in vitro cell culture to proliferate the cells in order to increase the yield from a single sample. This is important for clinical applications as one HLA-matched BM or UCB sample is usually all that is available. Initial experiments indicate that the analysis be carried out on day 8 and day 12 of culture. Once expansion is achieved over the 8 and 12-day period, longer culture periods can be added. Both the column flow through and the column bound cells (Lin⁻ and Lin⁺ cells in the case of the first iteration) will be collected and assayed for 1) FACS or immunocytochemistry for Flk-1, CD31, CD133, desmin, MyoD and muscle actin, 2) gene expression (using RT-PCR) for the same, and 3) in vitro capillary formation using a fibrin matrix for support. The column selection step will be modified to capture and remove non-EPC and pericytes (contaminating cells) as they arise in culture.
Development of an acute and chronic mouse model and testing of cell delivery systems. Hind limb ischemia will be generated in one leg of NOD/SCID or Rag-1 mice. The untreated limb will act as a control. Streptozotocin (STZ) treated animals (diabetic induction) that are also surgically treated to induce ischemia will also be tested. Diabetes is a major cause of limb ischemia with endogenous EPCs demonstrating reduced function in diabetic patients. A dual model of diabetes and ischemia will allow testing of whether exogenous EPCs and pericytes can function properly and lead to ischemic repair under sub-optimal tissue conditions, such as diabetes.
Functional testing of the animal model. Animals will be assessed for increased muscle mass and improved mobility. At the end of the analysis, animals will be sacrificed and using immunohistochemistry will be assessed for engraftment of human cells. Based on the results from the animal studies, cell culture and cell selection regimes will be optimized. Although immunocytochemistry and PCR will be used to expedite analysis of the different culture systems, ultimately engraftment will be used to eliminate or accept input populations.

### Preliminary Results:

Isolation and proliferation of EPCs from human UCB: Lin⁻ cells isolated using a negative selection column contained very few detectable EPCs, endothelial cells or pericytes. Lin⁻ cells consist of stem and progenitor cells of mesenchymal, endothelial and hematopoietic origin. Different growth factor regimes were tested for their ability to support the maintenance and expansion of stem and progenitor cells. FGF-4, SCF and Flt-31 growth factors (FSF1 medium) were found to give the best results.

OCT-4 and Nanog are important stem cell markers as they specify the multi-potential cells in ES cell colonies [Nichols, J. et al., Cell 95, 379-91 (1998); Boiani, M., et al, Genes Dev 16, 1209-19. (2002); Hattori, N. et al., J Biol Chem 279, 17063-9 (2004), Cell 113, 643-55 (2003); and Chambers, I. et al., Cell 113, 643-55 (2003)]. Lin⁻ cells at different stages of cell culture were tested for stem cell and non-blood cell gene and protein expression. Day 0 Lin⁻ cells after 8 days of growth in FSF1 medium could be induced to express embryonic and early stage tissue specific markers, eg. FLK-1, desmin and importantly, the embryonic stem cell markers Oct-4 and nanog. When FSF1 cultured Lin⁻ cells were transferred to bone differentiation medium, OCT-4 expression became down-regulated as expected for differentiating cells. The ability of the Lin⁻ cells to develop into endothelial or muscle cells using in vitro culture systems originally developed for embryonic stem cell differentiation was tested. With some modifications, both cell types were generated. Direct culture in differentiation medium (endothelial or muscle) without prior exposure to the FSF1 medium resulted in all cells dying, suggesting that culture in FSF1 medium is required for progenitor cell survival. Lin⁻ cells (day 0) tested negative for Flk-1 and CD31. Cells grown for 8 days in FSF1 medium became positive for Flk-1, an early marker of endothelial cells [40]. After 7 days in endothelial differentiation medium they remained Flk-1+ and CD31 negative. During the culture period cell morphology changed from round, non-adherent to elongated, adherent cells and Flk-1 expression was replaced by CD31 expression in about 50% of the cells after 14-21 days in culture. When the FSF1 grown cells were placed into 3-D cultures, which allow the formation of capillaries, cells migrated out of the colonies forming links to other colonies [41]. The cells would then curl under forming primitive tube-like structures. Day 8 Lin− FSF1 grown cells when placed into muscle differentiation medium resulted in the sequential activation of MyoD, muscle actin, and myosin heavy chain. The ability of the cells to contribute to vessel formation in NOD/SCID mice was also tested.

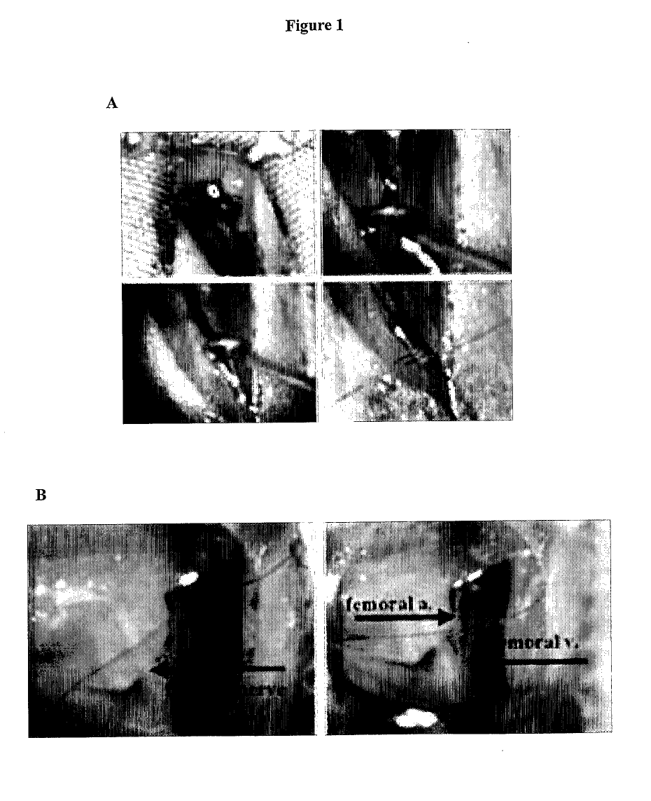

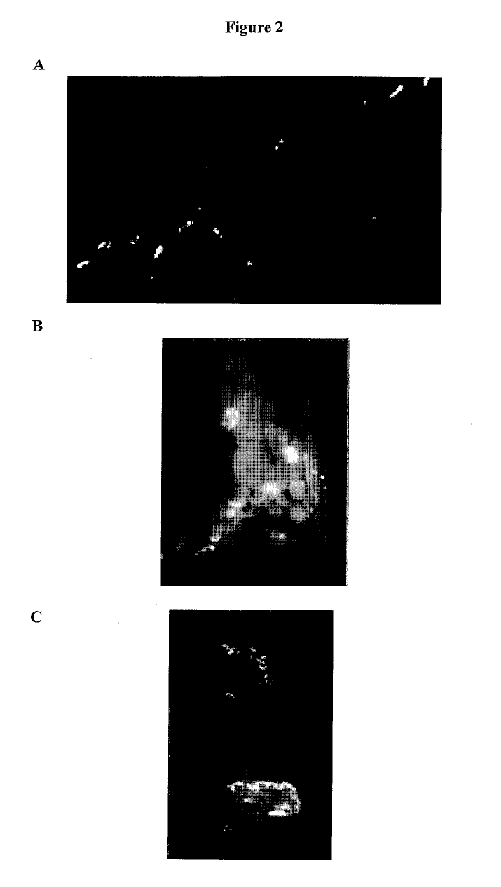

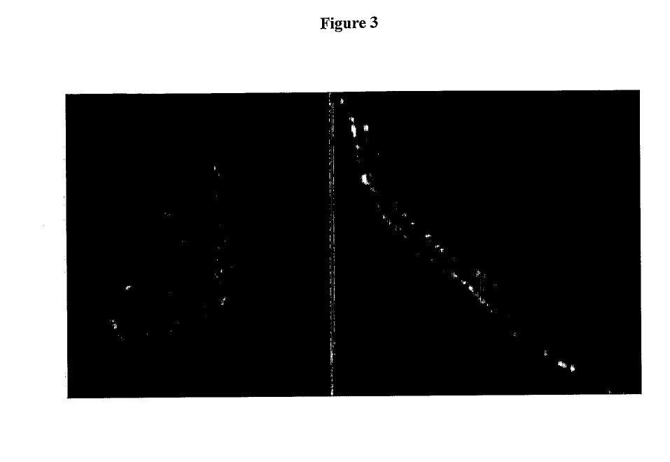

Ischemic mouse model: One million cells were injected into each of the adductor muscle and the gastrocnemius muscle at the time of surgery to induce ischemia (FIG. 1). Mice were assessed at day 7, day 14 and 8-weeks for the presence of human cells. The mice were assayed for human cells in general using an anti-human mitochondria antibody and to initially determine the presence of human cells. Positive mice were then assessed for human endothelial cells (using a human specific antibody to CD31) (FIGS. 2A, B and C) or muscle cells (using a human muscle actin specific antibody) (FIG. 3). Mice assessed at all three time points were positive for both human endothelial cells and smooth muscle cells. The efficiency of engraftment and differentiation was high (Table 1).

### Example 2

Improved enrichment and proliferation of mesenchymal cells, endothelial cells and pericytes from UCB and BM. In vitro cell expansion methods were used to increase the yield of precursor cells.
Isolation and culture of the input cell population. Cord blood cells are collected and processed by the Starch method (www.emmes.com). Donated research samples obtained with informed consent will be collected. Bone marrow cells will be purchased from Stem Cell Technologies (Vancouver, Canada). Stem/Progenitor cell populations will initially be isolated using the commercially available negative selection column Stem Sep column (Stem Cell Technologies, Vancouver, B.C.). The antibody cocktail is designed to remove differentiated cells (lineage positive) leaving behind stem and progenitor cells. The cells in the flow through are referred to as Lineage negative (Lin−) and contain hematopoietic stem cells, EPCs and mesenchymal cells. Column isolated cells will be cultured in FGF-4 (50 ng/mL), SCF (50 ng/mL) and FLT-3 ligand (50 ng/mL) supplemented in a serum free medium (BIT, STI)=FSF1 medium. These cytokines cause stem cell proliferation (Petzer, A. L., et al, J Exp Med 183, 2551-8 (1996); Yagi, M. et al. Proc Natl Acad Sci USA 96, 8126-31 (1999)). Cells will be seeded into cultures at 100,000 cells/ml. UCB/Lin⁻ cells in FSF1 culture develop mesenchymal cell properties including cells with characteristics of endothelial cells and muscle cells. BM will also be used as it has an advantage over UCB due to the ability to harvest cells directly from the patient in the case of non-diabetic patients. An in vitro culture system (serum free and feeder cell free) has been successfully designed that results in the extensive proliferation of mesenchymal cells and their derivatives as well as circulating EPCs. A 500-fold increase in endothelial cells was demonstrated. The method can generate sufficient cells for successful cell therapy for the treatment of PVD from a single cord blood unit. Both UCB and BM cells will be treated in an identical manner. BM contains many more mesenchymal cells and EPCs versus UCB, but expansion of cells for use in PVD cell therapy is preferred.

In the initial iteration of the culture system the media has been optimized for progenitor cell proliferation. Cultures will be depleted of specific cell populations that are deemed to interfere with the proliferation of progenitor cells at time=day 4 and day 8, with continued culture for 4 more days. At this time cells will be tested for mesenchymal, EC and muscle cell content. Specific cell populations that will be removed are: (a) mature blood cells, since these will arise in culture. Removing these will leave behind EPCs, pericytes and mesenchymal cells. Some blood cells are inhibitory, for example megakaryocytes express platelet factor-4 an inhibitor of VEGF and endothelial cell proliferation (Bikfalvi, A., Biochem Pharmacol 68, 1017-21 (2004). Ryo, R., et al.,. Leuk Lymphoma 8, 327-36 (1992)); (b) Mesenchymal cells are a mixed population and although some cells are supportive, osteoblasts and osteoclasts both present in blood and mesenchymal cell cultures, are inhibitors of endothelial growth through their production of pigment epithelial derived factor (PEDF) (Tombran-Tink, J. & Barnstable, C. J; Biochem Biophys Res Commun 316, 573-9 (2004). Cai, J., et al.,. J Biol Chem 281, 3604-13 (2006)). Inhibitory cells will be removed using cell-specific antibodies and flow cytometry or negative selection columns. Conversely a positive selection column will be tried to isolate mesenchymal cells, EPCs and pericytes, which will then be put into fresh medium for continued culture. Day 4, day 8 and day 12 populations with selection at day 4 and/or day 8 will be tested by: (i) Immunocytochemistry and PCR analysis: the expression of FLK-1, CD31, CD34, desmin, MyoD and muscle actin will be investigated; and (ii) In vitro capillary formation assay. When available, UCB samples from diabetic patients will be obtained and compared for their ability to generate EPCs and pericytes under the same conditions. (See, Madlambayan, Rogers, et al (2005), Exp't Hematology. 33, 1229-1239.)

### Example 3

PVD-mouse models. Two different mouse models will be developed and used to analyze the homing and engraftment potential of human EPCs and pericytes derived from UCB and BM. A surgical based hind-limb ischemia model in normal and diabetic mice will be developed. Mice will be generated with less severe injuries that are better reflective of the chronic human situation and the ability of cells to home and engraft in these animals will be established. Test animals will be used for toxicity and tumour studies followed by studies investigating engraftment levels.

A model of Mouse Hind Limb Ischemia Injury has been established and well characterized in the literature [43, 44, 45]. All surgical instruments are autoclaved and all procedures are done aseptically. 8-week old female/male NOD/SCID mice or Rag-1 mice will be used. (NOD/SCID mice are mildly diabetic at 4 months of age or older.) This is beyond the age of the mice used in these experiments, but urine glucose levels will be monitored in all NOD/SCID mice (not treated with STZ)). Mice are deeply anaesthetized with 2-3% Isoflurane. The area of incision is shaved, washed, disinfected with 70% ethanol first, and finally with surgical iodine (Betadine solution). The skin is incised at the right mid hind limb directly overlying the femoral artery. The artery is gently dissected from within the muscles and corresponding nerve and vein. The proximal end of the femoral artery close to the Inguinal ligament, and the distal fragment of the saphenous artery are ligated with 8-0 nylon suture. The whole portion of the artery between ligatures is cut and excised, while the branches are obliterated with an electric coagulator. Care is taken not to create any unnecessary mechanical or thermal damage to the surrounding tissues. The adductor muscle is exposed underneath the dissected insertion of the sartorius muscle. Then the adductor muscle and the gastrocnemius muscle-medial head are injected once in three spots each with human origin. The skin is closed with a 7-O-silk suture. Immediately after the operation the mice are injected i.p. 1.0 to 2.0 ml 0.9% saline, and s.c 0.025 ml/10 g Temgesic (2 mg/ml) as an analgesic. The animals recover in 5-15 minutes after the gas anaesthesia and start walking.

Postoperative care: All animals are given 1 ml novo-trimel/50 ml H₂O for 5 days. During this time, the animals are examined for any signs of infection daily. The mice are fully awake 5-10 min after the surgery and the heating lamp can then be taken away and the mice can be moved into separate cages. Any mouse showing any of the following symptoms; 20% loss in body weight, inactivity, problems breathing, no grooming, hunched posture, hypothermia, pinched face, and sunken eyes, would be immediately euthanized. The model is designed for maximum ischemic damage to the muscles affected for three reasons: 1. To make sure that the animals own cells do not compete with the human cells for repair so there is enough ‘space’ for the injected cord blood cells. 2. To check the survivability and differentiation potential of the cord blood cells injected. 3. To check the survivability/severity of the procedure on the mice. Only the femoral artery (from inguinal ligament to the bifurcation of the popliteal and saphena arteries) or a portion of the artery will be excised or the artery at respective points will be occluded in order to cause less ischemic damage to the muscles.
Diabetic/PVD Animal mode. Animals are treated with STZ (160 mg/kg single dose). Blood is tested for high glucose levels at 24 hours (220 mg/dL or higher is acceptable), and every 7 days after. Mice with high serum glucose levels at 7 days will be used to create hind-limb ischemia as described above. NOD/SCID mice and Rag-1 mice will be used to generate an ischemic model in the hind limb. Both strains can be used to generate multiple disease models since they tolerate surgery and human cell transplant well.

Both models will be tested in the following manner. Initially four populations of cells from BM and UCB will be tested separately: (i) Day 0 unfractionated cells (ii) day 0 Lin− cells (iii) Lin− day 8 FSF1 cells and (iv) mesenchymal cells. Initial data has demonstrated that Lin− day 8 FSF1 cells produced the highest yield of EPCs and muscle cells in vitro. Day 8 lin− FSF1 cell data will provide the base line to test cells. It is expected that in vitro conditions leading to improved yields of EPCs and pericytes will result in the improved correction of mouse hind limb ischemia. Input cells will require an appropriate environment to properly differentiate and integrate into vessels. The conversion rate of precursor cells (input cells) to mature endothelial and smooth muscle cells will be assessed by comparing the ratio of human cells present in the mouse tissue to human specific endothelial or muscle cells. Antibodies specific for human mitochondria will determine the number of human cells present and antibody to human CD31 (a mature endothelial marker) or smooth muscle actin will determine the number of cells that have differentiated. Cell position in the tissue and morphology will also be used as deterministic parameters for assessing positive results.

Delivery system for input cells. Once the optimal input cells are determined NOD/SCID transplantation will be used to test three different delivery systems. Initial studies discussed herein utilize the direct injection of cells into the muscle surrounding the occluded vessel during surgery. A more practical delivery system that is more clinically relevant can be developed. Research has determined that EPCs can home to the area of ischemia. This means that cells could be injected intramuscular in and around the area of ischemia. Multiple sites spaced equally apart could ensure the target area is reached. In order to track injection sites, animals with surgical occlusion will be left for 2-4 days post surgery and then cells will be delivered intramuscular. Since the leg area will have been shaved for surgery a 0.5 cm² (3×3) grid with 9 vertices will be set up. 1 μl of cells (300,000 cells) will be injected at each point. Mice will be assessed in the same way as described above.

EPCs are also found in the circulation and are capable of migrating to the affected area. This property will allow testing of cell delivery via the tail vein. Ischemic mice will be injected with 2 million cells in 500 directly into the tail vein 2 or 4 days post surgery. The advantage of this system is that one single injection can be carried out. Clinically it will be easier if the patient requires multiple injections, which may be required in recurring ischemia; for example, if the underlying cause of the ischemia, such as diabetes, is not controlled.

Quantitation of repair and function. Functional improvement can occur due to the direct contribution of the input cells to vessel structure or due to an indirect mechanism. Indirect mechanisms occur when the cells act as accessory cells providing growth factors or supportive functions that stimulate the endogenous endothelial and muscle progenitor cells to form vessels. Elucidating the dominant mechanism will allow manipulation of the in vitro proliferation conditions to produce the important cell types leading to improved engraftment levels and function.

The following approaches will be used: (i) High-resolution X-ray computed tomography (micro-CT) will be used to produce detailed three-dimensional images of the blood vessels. In order to obtain high resolution high doses of X-rays are required. This precludes scanning of live specimens. Therefore, micro-CT will be performed on animals at time=0, 1 day, 7 days, 14 days, 21 days and 28 days and 8-week post surgery. (ii) On a different set of mice treated in an identical manner, mobility will be assessed using an activity wheel (one mouse per cage). Readings of 24 hours of activity will be done at t=day 1 (the 24 hour period after surgery) day 7, 14, 21, 28 and 8-weeks. Staggered surgeries will allow testing of all animals using the two cages available. After the 8-week analysis animals will be sacrificed, tissue excised and tested for human cell content with muscle and endothelial specific antibodies (as above). (iii) Tissue/muscle cross-sections will be done in order to measure improvement in muscle mass. The diameter of muscle bundles (cross section) and the number of bundles at the midway point of the adductor muscle will be measured. Treated animals and controls will be compared. This will allow measurement of improvement due to direct and indirect effects of the cells. Control animals will be those that undergo surgery but do not receive cells, and since only one leg per animal is made ischemic the other can also act as a control.

### Example 4

### Treatment of Peripheral Vascular Disease (PVD) Using Endothelial, Smooth Muscle and Striated Muscle Precursor Cells Derived from Human Umbilical Cord Blood (UCB)

In this study the feasibility of delivering exogenous endothelial and muscle progenitor cells to the affected tissue in a PVD resulting in new vessel growth as well as contribution to the expansion of existing vessels is demonstrated. Combining surgical treatment with cell based therapy will greatly improve perfusion of the ischemic tissue.

### Material and Methods:

Blood collection and cell preparation: Written consent for collecting and processing umbilical cord blood was obtained at the time of registration for the study. Qualified hospital personnel, following protocols approved by the human ethics committee of the Mount Sinai Hospital and the University of Toronto, collected the cord blood at the time of delivery. The blood was collected with ACD to prevent coagulation. Pentastarch (Dupont) was added (1:5) and the sample spun at 50×g for 10 min at 10° C. to sediment the RBC. The leukocyte rich plasma was centrifuged at 400×g for 10 min at 10° C. to pellet the cells. The cell pellet was resuspended in IMDM containing 10% serum and mixed with an equal volume of cryoprotectant (20% DMSO/80% serum (heat inactivated/filtered), step frozen and stored in liquid nitrogen until required.
Stem cell enrichment: A negative selection column was used to remove mature cells (cells with the following markers: CD2, CD3, CD14, CD16, CD19, CD24, CD56, CD66 & glycophorin-A) as described by the manufacturer (Stem Cell Technologies, Vancouver, Canada). Lineage negative cells or the lineage positive cells were resuspended in the appropriate medium.
Cell Culture; Proliferation and Differentiation in FGF4/SCF/Flt3-ligand medium (FSF1): Proliferation: UCB-Lin^{neg} cells were seeded at 1×10⁵ cells/ml in StemSpan™ media (Stem Cell Technologies) containing Iscove's MDM, 1% BSA, 10 μg/ml insulin, 200 μg/ml human transferrin, 10⁻⁴ M 2-mercaptoethanol and 2 mM L-glutamine. The media was supplemented with 25 ng/ml stem cell factor (SCF; R&D Systems, Minneapolis, Minn.), 25 ng/ml Flt-3 ligand (FL; R&D Systems, Minneapolis, Minn.) and 50 ng/ml Fibroblast Growth Factor-4 (FGF-4; R&D Systems, Minneapolis, Minn.), 50 ng/ml heparin and 10 μg/ml low density lipoprotein (Sigma). Approximately 1.5 ml of the cell suspension was then placed into wells of a 24-well plate and maintained at 37° C. in a humidified atmosphere of 5% CO₂ in air for 8-days. 50% medium replacement occurs every 48 hours.

### In Vitro Differentiation of Lin-FSF1 Grown Cells to Endothelial Cells and Muscle Cells:

For cultures on chamber slides, cells were plated in M199 with serum (10%), supplemented with endothelial growth factor supplement (Sigma). Cells were fed twice per week by the removal of medium without the loss of cells [37].

For muscle, FSF1 grown cells were either transferred into 20% serum in DME (high glucose) for three weeks or transferred directly to muscle media at 37° C., normoxia conditions in αMEM+10% serum+Chick embryo extract (5%). Cells were cultured for 2-4 weeks and tested for muscle specific markers by immunocytochemistry.

Immunocytochemistry and Immunohistochemistry: After culture cells were washed in HBSS, fixed for 10 minutes with 10% formalin, washed and air-dried onto positive charged glass slides (Superfrost/plus, Fisher Brand, USA) and used immediately or stored at −86° C. Adherent cell cultures were grown on 8-well chamber slides (Labtek, Nalge, Nunc, USA), rinsed with HBSS, fixed as above and used immediately or air-dried briefly and stored at −86° C. For staining, all slides were immersed in HBSS for 5 minutes, blocked in 10% species appropriate serum/HBSS for 30-minutes, rinsed in HBSS (3×5 minutes). Primary antibody was added at 1/50 dilution-1/400 dilution in 1% serum/0.2% tritonx-100/HBSS at 4° C. for overnight. Slides were washed 5×5 minutes with 1% serum/HBSS. Secondary antibody was added at 1/400 dilution in 1% serum/HBSS and slides were incubated for 60 minutes at RT. Slides were washed 5×10 minutes in 1% serum/HBSS, and 1×HBSS.
Tissue processing for Immunocytochemistry: The tissues were fixed in 10% buffered formalin (Fisher Scientific) for 120 min—at 4° C. Washed in PBS (Phosphate Buffered Saline), storage in 70% Ethanol, dehydrated in graded ethanol series (80%-30 min, 95%-45 min, 2×100%/60 min), cleared in toluene—2×60 min, immersed in paraffin at 65° C. (Nr. I—30 min, Nr. II—45 min, Nr. III—60 to 120 min), embedded into paraffin blocks, cut on a microtome into 5 μm sections, put on Fisherbrand Superfrost Plus microscope slides, and let dry overnight. Sections were deparaffinized 2×5 min in xylenes and rehydrated through graded ethanol rinsed in deionized water, and washed in PBS for 5 min Blocked nonspecific binding was blocked with 10% serum in PBS containing 0.1% Triton X-100 (Sigma) for 240 min at room temperature, red fluorescent background dye Chicago Sky Blue, (Sigma) was applied. There was a brief wash in PBS after blocking and the fluorescent dye steps. Primary antibody was applied (solution 1:50) for overnight incubation at 4° C. Untreated mouse tissue sections were used as negative control. Sections with omitted primary or secondary antibody were used as another type of negative control in each experiment. Washed 5×15 min in PBS. Secondary antibody, was applied at 1:200 dilution for 60 min at room temperature. Washed 6×15 min in PBS. DAPI (Sigma) staining (nucleus) at 2 μg/mL for 2 min was followed by 5 min wash in PBS, and mounting in 50% glycerol in PBS with DABCO (Sigma) at 100 mg/mL.

Slides were examined on a Zeiss Axioplan Photomicroscope equipped with epifluorescent ultraviolet light and corresponding excitation and barrier filters. Pictures were taken on a Nikon Coolpix4500 digital camera or on a Delta Vision wide-field, optical sectioning microscope workstation capable of recording three-dimensional images of fluorescently labeled specimens (Issaquah, Wash.). The station includes: an Olympus IX-70 inverted fluorescence microscope with custom optical filters, and precision XYZ motorized stage, O2 silicon Graphics computer work station with image collection and deconvolution software.

Antibodies used: Mouse anti-human mitochondria IgG1 monoclonal antibody (Chemicon), anti-CD31 (Chemicon), anti smooth muscle actin (human) and anti human muscle actin (Chemicon), Anti-goat, anti-rabbit and anti-mouse secondary antibodies (Chemicon and Jackson, ImmunoResearch).

Hind Limb Ischemia procedure in Mouse Peripheral Vascular Disease Model: A model of Mouse Hind Limb Ischemia Injury have been established and well characterized in the literature [43-45]. All surgical instruments are autoclaved and all procedures are done aseptically. Eight week old female/male NOD/SCID mice were used, which were deeply anaesthetised with 1-2% Isoflurane. The area of incision was shaved, washed, disinfected with 70% ethanol first, and finally with surgical iodine (Betadine solution). The skin was incised at the right mid hind limb directly overlying the femoral artery. The artery was gently dissected from within the muscles and corresponding nerve and vein. The proximal end of the femoral artery close to the Inguinal ligament, and the distal fragment of the saphenous artery were ligated with 8-0 nylon suture. The whole portion of the artery between ligatures was cut and excised, while the branches are obliterated with an electric coagulator. Care was taken not to create any unnecessary mechanical or thermal damage to the surrounding tissues. (See FIGS. 1A, B and 5A, B.)

The adductor muscle was exposed underneath the dissected insertion of the sartorius muscle. Then the adductor muscle and the gastrocnemius muscle-medial head were injected once in three spots each with human origin Cord Blood Cells. The skin was closed with a 7-O-silk suture. Immediately after the operation the mice were injected i.p. 1.0 to 2.0 ml 0.9% saline, and s.c 0.025 ml/10 g Temgesic (2 mg/ml) as an analgesic. The animals recovered in 5-15 minutes after the gas anaesthesia and start walking.

Postoperative care: All animals were given 1 ml novo-trimel/50 ml H₂O for 5 days. During this time, they were examined for any signs of infection daily. The mice were fully awake 5-10 min after the surgery and the heating lamp was then taken away and the mice were moved into separate cages. Any mice showing distress such as breathing problems or weight loss exceeding 25% were immediately euthanised. Any mouse showing any of the following symptoms; 20% loss in body weight, inactivity, no grooming, hunched posture, hypothermia, pinched face, and sunken eyes, were immediately euthanized.
Cells used for Transplant: three populations of cells from UCB were tested separately: (i) Day 0 unfractionated cells (ii) day 0 Lin− cells (iii) Lin− day 8 FSFL cells

### Quantification of the Vascular Recovery

Laser Doppler Imaging followed by Micro Computed Tomography (MicroCT) was used to assess the mice post treatment. For Laser Doppler Imaging (LDI) the animals were anesthetized, placed on a heating pad and scanned three times to ensure that stabilized measurements would be taken.

After the procedure the animals were terminated and prepared for microCT using whole body perfusion. The hind limb muscles were excised and analysed. This analysis was done by the mouse imagining center in Toronto (www.mouseimaging.ca). (ii) After each time point animals were sacrificed, tissue excised and tested for human cell content with muscle and endothelial specific antibodies (as above). (iii) Tissue/muscle cross-sections were done in order to measure improvement in muscle mass. The diameter of muscle bundles (cross section) and the number of bundles at the midway point of the adductor muscle were measured. Following micro CT the tissue was collected and used for IHC analysis. The micro-CT preparation did not interfere with the antibodies used for IHC. The analysis allowed the comparison of the levels of engraftment of the UCB cells with the levels of angiogenesis/arteriogenesis and this was related to levels of blood flow observed by Laser Doppler. This is important for establishing a minimum cell dose that is required to obtain a statistically significant improvement.

Treated animals and controls were compared. Control animals are those that undergo surgery but do not receive cells, and since only one leg per animal is made ischemic the other can also act as a control. The data indicates that the unfractionated UCB cells do not engraft. These cells also act as a control for the antibodies and as a base to compare the day 8 FSF1 cells (cultured UCB cells).

### Results

UCB Lineage Minus Cells were Capable of Differentiating into Endothelial Cells and Muscle Cells:

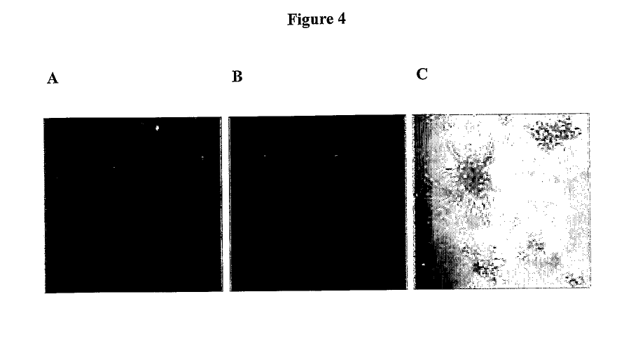

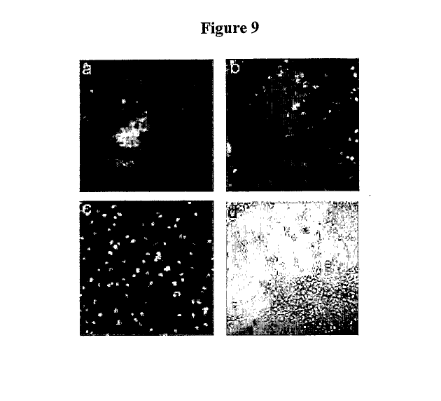

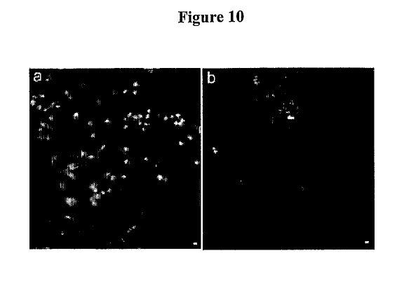

Using a negative selection column a population enriched for stem cells from Umbilical Cord Blood (Lin− cells) were isolated. Lin− cells (day 0) tested negative for Flk-1, CD31, Desmin and MyoD. After 8 days of growth in FGF/SCF/FLT3ligand (FSF1) supplemented medium the cells were transferred to in vitro differentiation medium. Depending on the specificity of the differentiation medium, cells either expressed endothelial markers (FLK-1, CD31) or muscle markers (desmin and MyoD). After 7 days in endothelial differentiation medium they remained Flk-1positive and CD31 negative but with prolonged culture Flk-1 was down regulated as expected and CD31 was evident (FIG. 4A,B,C and FIG. 9a,b,c). Day 8 Lin− FSF1 grown cells when placed into muscle differentiation medium resulted in the sequential activation of MyoD, muscle actin, and myosin heavy chain (FIG. 4B, FIG. 10a,b).

UCB Lineage minus cells can contribute to vessel formation in NOD/SCID mice: A mouse model of hind limb ischemia was successfully created in NOD/SCID mice which allowed the extent of the damage done to the mice through removal and cauterization of selected vessels in the hind limb to be controlled. The model was designed for maximum ischemic damage to the muscles for three reasons:
1. To make sure that the animal cannot recover by itself so there is no competition from the endogenous cells.
2. To check the survivability and differentiation potential of the cord blood cells injected.
3. To check the survivability/severity of the procedure on the mice.

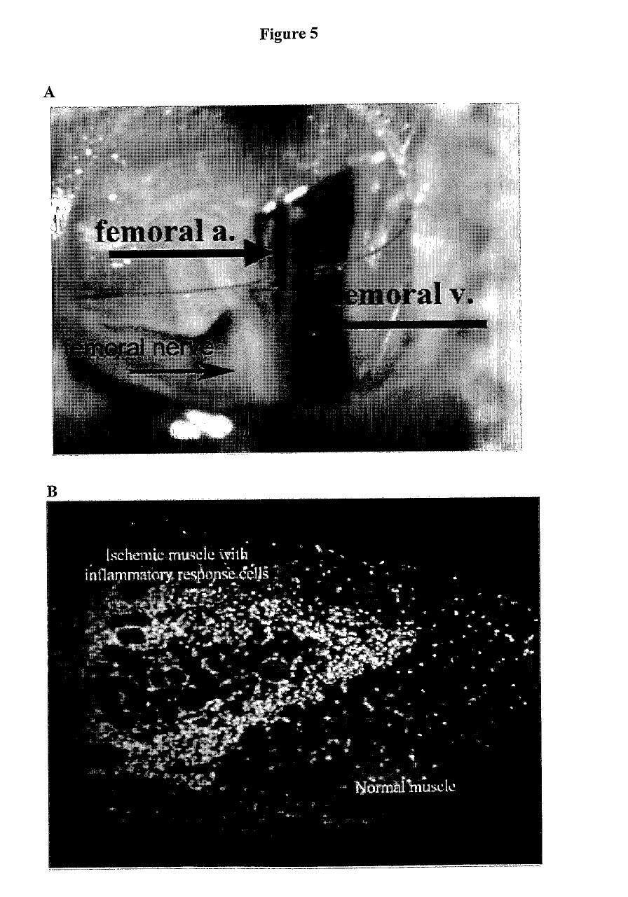

The femoral artery was removed and the attached vessels cauterized (FIG. 5A). This caused localized ischemia causing a degeneration of the muscle tissue (FIG. 5B).

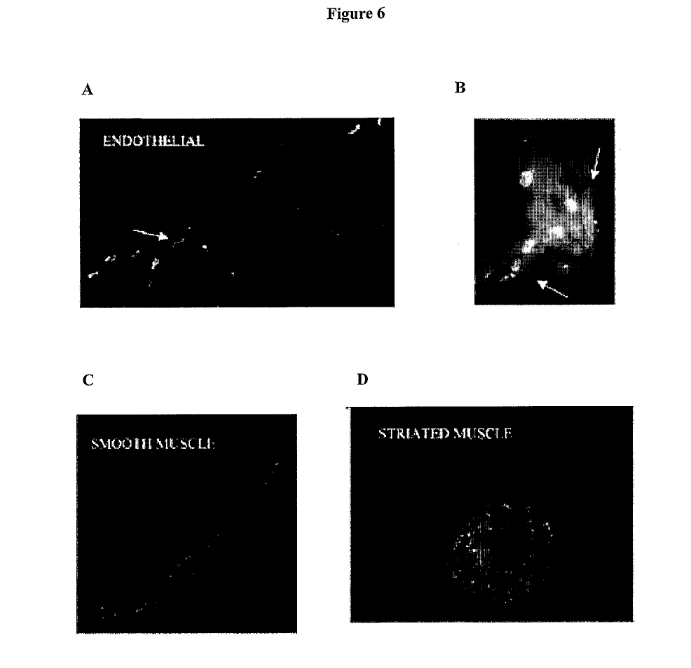

The UCB Lin^{neg} FSF1 grown cells when injected into the muscle had a high engraftment rate in the ischemic limb contributing to both the endothelial and the smooth muscle components of vessels. Furthermore a small but significant contribution to the striated muscle was seen. (See FIGS. 6A, B, C and D.)

### Endothelial Cells:

1 million Lin-FSF1 cells were injected into each of the adductor muscle and the gastrocnemius muscle at the time of surgery. Mice were assessed at day 7, day 14 and 4-weeks for the presence of human cells using an anti-human mitochondria antibody. Positive mice were then assessed for the ability of the FSF1 cells to differentiate in vivo into human endothelial cells using a human specific antibody to CD31. Mice assessed at all three time points were positive for human endothelial cells. Antibodies specific for human mitochondria were used to determine the number of human cells present and antibody to human CD31 was used to determine the number of cells that have differentiated. This allowed the determination of the frequency of engrafting cells and the frequency of differentiation into mature endothelial cells (FIG. 6A,B). The efficiency of engraftment and differentiation was high; 1-23% of infused cells stained positive for CD31. FSF1 cells produced 100× more engrafted cells (CD31) when compared to Lin− cells (uncultured).

### Muscle Differentiation:

The reduction in blood flow generated by the removal of the femoral artery induced new vessels growth and the enlargement of existing vessels as the limb compensate. Whether the FSF1 grown cells were capable of contributing to the smooth muscle portion of larger vessels during their enlargement process was investigated. Using a human specific antibody against smooth muscle actin, smooth muscle cells in large vessels in the mouse limb that were derived from the human UCB cells were detected. (FIG. 6C). The ischemic limb demonstrated regeneration of the striated muscle. Regenerating muscle cells have center nuclei. In FIG. 6D one regenerating area is positively detected by an antibody to human muscle actin. Table 1 illustrates the frequency of engraftment of human cells and the frequency of differentiation of the input cells to mature endothelial cells. In some vessel segments the human contribution was very high.

Laser Doppler and microCT:

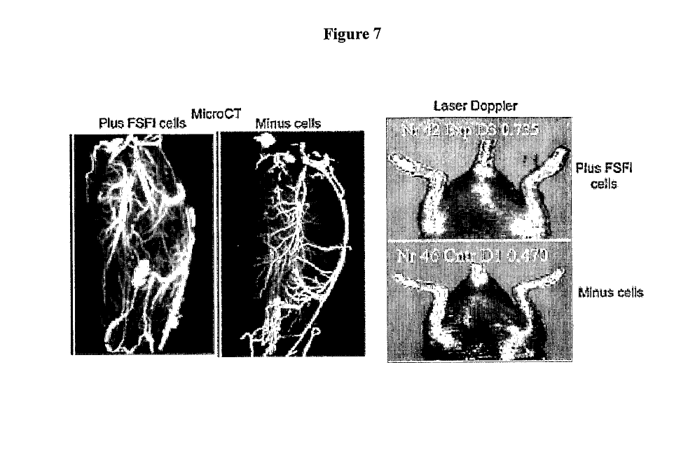

Mice at 4 weeks post surgery were subjected to Laser Dopler Image analysis followed by processing for MicroCT. Laser Doppler Imaging (LDI) is a widely used technique used to assess superficial blood flow that can reflect the degree of recovery after the ischemia. This allowed for a direct assessment of the ability of the FSF1 grown cells to integrate into the existing vasculature and recreate the vascular network. Since LDI cannot tell the change in the vascular bed volume in the recovery after the ischemia and since most of that volume change occurs in the medium-size vessels (collateral circulation), microCT is used to measure this. Micro CT is more relevant for the recovery after ischemia than the capillary density [47]. MicroCT measurements in the ischemic leg allowed for a visual comparison between animals treated with cells versus those left untreated. Both microCT and laser doppler demonstrated an observable increase in new vessel formation (MicroCT) and increased blood flow (LDI) compared to ischemic limbs not infused with test cells (FIG. 7).

### Engraftment of Human Cells to Mouse Blood Vessels Did not Occur Due to Fusion:

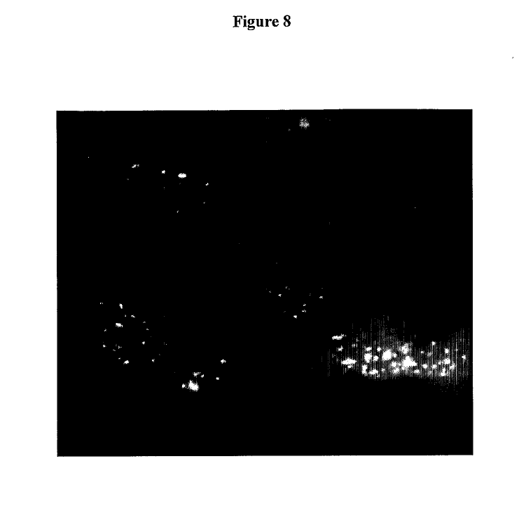

It is possible that the engraftment of the human cells is due to fusion with the murine cells. Although it was demonstrated that the human UCB cells are capable of integrating into existing vessels and contributing to new vessels and regenerating muscle fibers. Fluorescent in situ hybridization (FISH) with mouse and human centromeric probes were used to determine if fusion is occurring. Immunocytochemistry was carried out on sections of muscle from ischemic mice treated with cells from Lin− FSF1 grown cells using a human specific antibody to human mitochondria. Sections positive for human cells were reanalysed by FISH. Using a deconvolution microscope to take optical sections it was determined that no fusion occurred. Cells contained either human or mouse chromosomes but not both. Cells positive for human mitochondria were also positive for human chromosomes. No cells contained human mitochondria and mouse chromosome confirming that fusion did not occur (FIG. 8).

### Discussion

The application of a single cell type or individual growth factor may result in partial repair but the goal should be for more complete care. In this study it was demonstrated that unmanipulated UCB have low levels of EPCs and mesenchymal cells but the culture system is capable of supporting the proliferation of multi-potential progenitor cells that are capable of further differentiation in vivo into endothelial cells, smooth muscle cells and striated muscle [1]. The UCB cells lead to an increase in angiogenesis (micro CT) and to an improvement in blood flow (Laser Doppler) and the IHC demonstrated that the UCB cells contributed by differentiating into endothelial cells, smooth muscle and striated muscle.

Clinical trials have been reported. A patient with an ischemic toe ulcer and rest pain was given an injection of EPCs into the calf muscle and 4 weeks after treatment she could walk 10 minutes and had an ankle-brachial index double that of pre-treatment. An angiogram demonstrated an increase in vascularization of the ankle [22]. Cell-free therapy using exogenous growth factors has also been explored. Exogenous growth factors can stimulate the endogenous cells to contribute to the repair of the ischemic tissue. This therapy is limited if the endogenous cells are not available due to extensive damage of the tissue or are not responsive, as may be the case with diabetic patients.

Diabetic patients with ischemia present a unique set of hurdles. Schatteman [19] demonstrated that exogenous EPCs from non-diabetic mice were capable of contributing to vasculogenesis in diabetic mice with PVD16. EPCs taken from diabetic patients did not differentiate in vitro into mature endothelial cells as well as those from non-diabetic patients. Other studies have demonstrated a reduced number of EPCs from diabetic patients versus non-diabetic patients [17-19]. Revascularization by exogenous healthy cells (human) has been demonstrated in diabetic mice with ischemia suggesting that the reduced wound healing is due to the inability of the EPCs and not due to the surrounding tissue to support regeneration. Therefore the transplantation of cells from a healthy donor will contribute to the repair of a diabetic foot ulcer. The work supports the use of UCB cells as a donor source.

### Example 5

This example describes methods for the preparation of a cellular product with an expanded population of CD45+ multipotential cells from human UCB. These cells are non-adherent at the time of isolation. After 8 days of culture in a defined medium, the cellular product can be differentiated into mesenchymal, endothelial and muscle cells.

UCB-derived CD45-positive/lineage-negative (CD45+/lin−) cells are expanded in a medium designed to promote stem cell proliferation without differentiation and the resulting cell population and its in vitro differentiation potential is characterized. In particular, UCB-derived lin− cells were cultured in a serum-free medium supplemented with stem cell factor (SCF), Flt-3 ligand (FL) and fibroblast growth factor-4 (FGF-4). Cells were maintained at 37° C. in a humidified atmosphere of 5% CO₂ in air for 8 days. Fifty percent medium replacement occurred every 48 hours. The phenotype and cell expansion was assessed at culture termination. The final cell product was also assessed for its ability to differentiate into endothelial and muscle cells in vitro.

### Results:

### Expansion and Characterization of Final Cell Product

The culture conditions resulted in an increase in the absolute number of CD45+ and CD34+ cells over the 8 day culture period. Phenotypic analysis and cell expansion data from the cell culture are summarized in Tables 2 and 3, respectively.

### Multi-Lineage Potential of the Final Cell Product

The final cell product could be differentiated along multiple cellular pathways in vitro through culture in specific differentiation media (see below). The starting lin− cells were also tested for their ability to differentiate into non-blood cell types. In all cases, the Day 0 lin− cells died in endothelial, muscle, bone or neural differentiation medium. In conclusion the multipotential cell properties of the multipotent cell product occur after culture.

Differentiation into Endothelial Cells

The mulitpotent cell product was cultured in endothelial differentiation medium (M199/10% FBS/endothelial growth factor supplement) for 1-2 weeks then examined by immunocytochemistry for the expression of endothelial markers. Prior to culture in endothelial differentiation medium, the multipotent cell product itself expressed Flk-1 (FIG. 9a). After 1 week in endothelial differentiation medium, the cells expressed the mature endothelial marker CD31 (FIG. 9b) and after 2 weeks in endothelial culture 100% of the cells expressed CD31 (FIG. 9c). Uncultured UCB-derived lin− cells did not survive if cultured directly in endothelial differentiation medium. The multipotent cell product was also cultured in a 3-dimensional fibrin matrix which supports the growth of 3-dimensional capillary structures. After 3-4 weeks, primitive vessel-like structures could be observed in culture (FIG. 9d).

Differentiation into Muscle Cells

Desmin, an early muscle marker, was detectable in the multipotent cell product, as determined by RT-PCR analysis. Culture of the multipotent cell product in muscle differentiation medium for 2 weeks resulted in the expression of MyoD, as determined by RT-PCR. The expression of muscle specific actin protein was detectable by immunocytochemistry when the multipotent cell product was differentiated in muscle differentiation medium (FIG. 10a). The representative result shown is from the multipotent cell product cultured in reduced serum (1%) and normoxia conditions. Myosin heavy chain expression was observed in the multipotent cell product muscle-differentiated cells. Specifically, myosin heavy chain was expressed in the muscle cells that had undergone fusion whereas individual cells remained negative for myosin heavy chain (FIG. 10b). This is similar to normal muscle development. Uncultured UCB-derived lin− cells did not survive if cultured directly in muscle differentiation medium.

### Conclusions:

The culture of UCB-derived CD45+/lin− cells in a medium containing exogenous SCF, FL and FGF results in the expansion of CD34+ and CD45+ cells. The expanded cell product is capable of differentiation into endothelial and muscle cells.

### Example 6

### Clinical Assessment of a Cell Product in Patients with Critical Limb Ischemia

### Preparation of Cell Product

The starting material for the clinical cell product will be a UCB unit obtained from public UCB banks compliant with the quality standards of FACT/NETCORD. Once an UCB unit has been identified for potential use, a sample of that unit will be tested to verify HLA type and cell viability. The UCB unit will be obtained as cryopreserved, red blood cell depleted, volume reduced samples. The UCB unit selected for the manufacture of the cell product will have a 6/6 HLA-A and B (intermediate resolution) and DRB1 (high resolution) match to the intended recipient. Further, the UCB unit selected must contain a minimum total nucleated cell count of 650 million cells post-processing (ie, before cryopreservation) to ensure a sufficient cell dose in the final cell product.

A culture medium that will be used to prepare the clinical cell product is StemSpan® SFEM™ medium supplemented with 25 ng/mL stem cell factor (SCF), 25 ng/mL Flt-3 ligand (FL), 50 ng/mL fibroblast growth factor-4 (FGF), 50 ng/mL heparin and chemically defined lipids (FSF1). An enriched population of hematopoietic stem cells (lin− cells) is isolated from the thawed UCB unit using the StemSep® Human Hematopoietic Progenitor Cell Enrichment Kit. The lin− enrichment is achieved through negative selection, i.e., the removal of lineage-positive (lin+) cells. The process is described briefly below.

### Labelling of Lin+ Cells:

1. StemSep® Progenitor Enrichment Cocktail is added to the cells at 100 μL/mL cells.
2. The cells/cocktail mixture is incubated at room temperature for 15 minutes.
3. Magnetic Colloid (60 μL/mL cells) is added to the cell suspension and mixed well.
4. The cells/cocktail/colloid mixture is incubated at room temperature for 15 minutes.

### Preparation of Separation Column:

1. The column is placed in the magnet and assembled as illustrated in the manufacturer's procedure.
2. The column is then primed with 21 mL HBSS.

### Depletion of Lin+ Cells:

1. The labelled cell suspension is loaded onto the column and allowed to pass through by gravity feed. The labelled lin+ cells are retained by the column.
2. The column is washed with HBSS/2% plasbumin, and the unlabelled lin− cells, which are not bound to the column, are eluted in HBSS/2% plasbumin. This unbound fraction is enriched for hematopoietic stem and progenitor cells.
3. The enriched lin− population is pelleted, resuspended in 1-2 mL culture medium and counted.
4. The cell density is adjusted to 5×10⁴ cells/mL in the culture medium prior to the initiation of the expansion culture.

### Initiation of Expansion Culture

The enriched lin− cells are seeded into a 12-well culture dish at 5×10⁴ cells/mL, 1 mL per well, in FSF1 medium. The culture dish is placed into a cell culture incubator and maintained at 37° C. in a humidified atmosphere of 5% CO₂ in air. A 50% media exchange is performed on days 2, 4, and 6 of culture.

### Harvest of Multipotent Cells

After the 8-day culture period, the tissue culture dish is transferred to a biosafety cabinet. Cells are gently resuspended in the day 8 culture medium and transferred to a 50 mL sterile centrifuge tube. After transfer, the wells are rinsed with 1 mL sterile HBSS to collect any residual cells. The HBSS rinse is pooled with the initial cell suspension, the cells are counted then pelleted at 400 g for 3 minutes. The supernatant is decanted and the cell pellet is resuspended either in PBS/1% Plasbumin, if they are to be used within 48 hours, or cryopreservation medium if they are to be cryopreserved prior to use. For cryopreservation, the cells are resuspended in 1 mL HBSS/10% Plasbumin and cooled to 4° C. Freezing medium consists of 1 mL 50:50 Dextran (10%):DMSO, also cooled to 4° C. The freezing medium is added dropwise to the cells, then cooled at a rate of 1° C./minute to −90° C. and transferred to liquid nitrogen until required. Selected properties of the cell product are shown in Tables 2 and 3.

### Trial Protocol Summary

A clinical trial will be conducted in patients with critical limb ischemia who are not candidates for non-surgical or surgical revascularization. The objectives of the trial are to assess the safety of the cell product in patients with critical limb ischemia to assess preliminary efficacy of the product in increasing blood flow in the ischemic limb through improvements in: the ankle-brachial index (ABI); pain at rest; pain free walking time; ulcer healing; incidence of amputation; transcutaneous oxygen pressure; and digital subtraction angiography

The cell product will be administered via intramuscular injection into the affected limb. The cell product (cell dose≧2.0×10⁷ CD34+ cells) is prepared from an allogeneic UCB unit with a 6/6 HLA match to the intended recipient. The UCB unit will be expanded using the process described above. Immunosuppressive therapy will be given to prevent rejection of the cell product.

The study will be open to the following subjects with documented critical limb ischemia: male or female subjects 18-80 years old; critical limb ischemia with documented pain at rest, nonhealing ulcers or both; an ankle brachial pressure index<0.6 in the affected limb on two consecutive examinations done at least one week apart; and existence of a suitable UCB unit with a 6/6 HLA match to the patient. Patients will be excluded based on the following criteria: poorly controlled diabetes mellitus (HbA1>8%); underlying retinal pathology based on a fundoscopic examination; comorbid conditions other than critical limb ischemia that limits the patient's ability to exercise; current or history of malignant disorder in the past 5 years; suspicion of malignancy after cancer screening; inflammatory or progressive fibrotic disorder; renal insufficiency or proteinuria; women of child bearing potential; and pregnant or breast feeding women.

Tissue typing will be performed on eligible patients to determine the HLA status of the patient so that a search for suitably matched UCB can be instituted. The criteria for a UCB unit being acceptable for use in the study are: 6/6 HLA match; a minimum total nucleated cell count of 650 million viable cells post-processing (i.e., before cryopreservation); and adequate donor screening. If a patient is eligible for the study, preparation of the cell product will commence and the product will subsequently be administered. The cell product will be administered by intramuscular injection into the ischemic leg, with a total injection volume of ˜3 mL. The delivery location will be standardized as follows. The study drug will be administered as 20×150 μL injections, separated by 1.5 to 2.0 cm both anteriorly and posteriorly.

The following endpoints will be assessed: change in ankle/brachial index; change in pain free walking time; change in laser Doppler flow; change in tissue perfusion; ulcer healing; and, number of amputations.

TABLE 1
Frequency of engraftment and differentiation of UCB cells

| | α-mitochondria | α-CD31 | | |
|---|---|---|---|---|
| #cell in 600K | [cells/muscle] | [cells/muscle] | | % |
| Adductor-1 | 5,000 | 3,000 | [0.5%] | 60% |
| Gastrocnemius-1 | 12,000 | 6,000 | [1%] | 50% |
| Adductor-2 | 140,000 | 140,000 | [23%] | 100% |
| Gastrocnemius-2 | 75,000 | 75,000 | [12.5%] | 100% |

Human mitochondria positive cells were counted and used to determine engraftment rate. Human CD31 positive cells define mature endothelial cells and from this the frequency of differentiation could be calculated as a percentage of total cells infused or as a percent of engrafted human cells

TABLE 2
Phenotypic Analysis of Cells Pre- and Post- Culture

% positive (±STD)

| Cell | Day 0 | Day 8 | |
|---|---|---|---|
| Phenotype | (lin⁻ enriched cells) | (multipotent cells) | |
| CD45⁺ | 80 (±22) | 99 | (±6) |
| (n = 9) | | | |
| CD34⁺ | 68 (±19) | 74 | (±4) |
| (n = 12) | | | |
| CD34⁺/CD38⁺ | 35 (±31) | 60 | (±17) |
| (n = 6) | | | |
| CD34⁺/CD38⁺ | 33 (±22) | 14 | (±5) |
| (n = 6) | | | |
| CD34⁺/CD38⁺ | 23 (±14) | 19 | (±11) |
| (n = 6) | | | |
| CD34⁺/CD38⁺ | 8 (±6) | 6 | (±3) |
| (n = 6) | | | |
| Viability | | 95 | (±10) |
| (n = 8) | | | |

TABLE 3
Expansion Data for Selected Cell Types

| | Mean Cell Density (±STD) | | |
|---|---|---|---|
| | (cells/mL) | | Mean Fold |
| | Day 0 | Day 8 | Expansion |
| Cell Type | (lin⁻ enriched cells) | (multipotent cells) | (±STD)* |
| Total | 100,000 | 960,000 (±58,000) | 9.6 (±5.8) |
| Nucleated | | | |
| Cells (n = 10) | | | |
| CD45⁺ | 80,000 (±19,000) | 960,000 (±56,000) | 14.5 (±18) |
| (n = 9) | | | |
| CD34⁺ | 68,000 (±13,000) | 710,000 (±43,000) | 10.4 (±21) |
| (n = 7) | | | |

*Calculated as the mean fold expansion from individual experiments.

While the present invention has been described with reference to what is presently considered to be a preferred embodiment, it is to be understood that the invention is not limited to the disclosed embodiment. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### PUBLICATION CITATIONS

1. Rogers, I. et al. Identification and analysis of in vitro cultured CD45-positive cells capable of multi-lineage differentiation. Exp Cell Res 313, 1839-52 (2007).
2. Staudacher, D. L., Preis, M., Lewis, B. S., Grossman, P. M. & Flugelman, M. Y. Cellular and molecular therapeutic modalities for arterial obstructive syndromes. Pharmacol Ther 109, 263-73 (2006).
3. Rice, T. W. & Lumsden, A. B. Optimal medical management of peripheral arterial disease. Vasc Endovascular Surg 40, 312-27 (2006).
4. Sivan-Loukianova, E., Awad, O. A., Stepanovic, V., Bickenbach, J. & Schatteman, G. C. CD34+ blood cells accelerate vascularization and healing of diabetic mouse skin wounds. J Vasc Res 40, 368-77 (2003).
5. Eckman, M. H. et al. Foot infections in diabetic patients. Decision and cost-effectiveness analyses. Jama 273, 712-20 (1995).
6. CDC.
7. Layery, L. A. et al. Risk factors for foot infections in individuals with diabetes. Diabetes Care 29, 1288-93 (2006).
8. Feener, E. P. & King, G. L. Vascular dysfunction in diabetes mellitus. Lancet 350 Suppl 1, SI9-13 (1997).
9. Duh, E. & Aiello, L. P. Vascular endothelial growth factor and diabetes: the agonist versus antagonist paradox. Diabetes 48, 1899-906 (1999).
10. Marfella, R. et al. Expression of angiogenic factors during acute coronary syndromes in human type 2 diabetes. Diabetes 53, 2383-91 (2004).
11. Sasso, F. C. et al. Increased vascular endothelial growth factor expression but impaired vascular endothelial growth factor receptor signaling in the myocardium of type 2 diabetic patients with chronic coronary heart disease. J Am Coll Cardiol 46, 827-34 (2005).
12. Reiber, G. E., Pecoraro, R. E. & Koepsell, T. D. Risk factors for amputation in patients with diabetes mellitus. A case-control study. Ann Intern Med 117, 97-105 (1992).
13. Pecoraro, R. E., Reiber, G. E. & Burgess, E. M. Pathways to diabetic limb amputation. Basis for prevention. Diabetes Care 13, 513-21 (1990).
14. Aiello, L. P. et al. Vascular endothelial growth factor in ocular fluid of patients with diabetic retinopathy and other retinal disorders. N Engl J Med 331, 1480-7 (1994).
15. Brem, H., Sheehan, P., Rosenberg, H. J., Schneider, J. S. & Boulton, A. J. Evidence-based protocol for diabetic foot ulcers. Plast Reconstr Surg 117, 193S-209S; discussion 210S-211S (2006).
16. Schmieder, R. E. Endothelial dysfunction: how can one intervene at the beginning of the cardiovascular continuum? J Hypertens Suppl 24, S31-5 (2006).
17. Fadini, G. P. et al. Number and function of endothelial progenitor cells as a marker of severity for diabetic vasculopathy. Arterioscler Thromb Vasc Biol 26, 2140-6 (2006).
18. Klibansky, D. A., Chin, A., Duignan, I. J. & Edelberg, J. M. Synergistic targeting with bone marrow-derived cells and PDGF improves diabetic vascular function. Am J Physiol Heart Circ Physiol 290, H1387-92 (2006).
19. Schatteman, G. C., Hanlon, H. D., Jiao, C., Dodds, S. G. & Christy, B. A. Blood-derived angioblasts accelerate blood-flow restoration in diabetic mice. J Clin Invest 106, 571-8 (2000).
20. Ferrara, N. & Kerbel, R. S. Angiogenesis as a therapeutic target. Nature 438, 967-74 (2005).
21. Brem, H., Young, J., Tomic-Canic, M., Isaacs, C. & Ehrlich, H. P. Clinical efficacy and mechanism of bilayered living human skin equivalent (HSE) in treatment of diabetic foot ulcers. Surg Technol Int 11, 23-31 (2003).
22. Kohlman-Trigoboff, D., Lawson, J. H. & Murphy, M. P. Stem cell use in a patient with an ischemic foot ulcer: a case study. J Vasc Nurs 24, 56-61 (2006).
23. Asahara, T. et al. Isolation of putative progenitor endothelial cells for angiogenesis. Science 275, 964-7 (1997).
24. Hristov, M. & Weber, C. Endothelial progenitor cells: characterization, pathophysiology, and possible clinical relevance. J Cell Mol Med 8, 498-508 (2004).
25. Rehman, J., Li, J., Orschell, C. M. & March, K. L. Peripheral blood “endothelial progenitor cells” are derived from monocyte/macrophages and secrete angiogenic growth factors. Circulation 107, 1164-9 (2003).
26. Hakuno, D. et al. Bone marrow-derived regenerated cardiomyocytes (CMG Cells) express functional adrenergic and muscarinic receptors. Circulation 105, 380-6. (2002).
27. Wakitani, S., Saito, T. & Caplan, A. I. Myogenic cells derived from rat bone marrow mesenchymal stem cells exposed to 5-azacytidine. Muscle Nerve 18, 1417-26. (1995).
28. Prockop, D. J. Marrow stromal cells as stem cells for nonhematopoietic tissues. Science 276, 71-4. (1997).
29. Theise, N. D. et al. Derivation of hepatocytes from bone marrow cells in mice after radiation-induced myeloablation. Hepatology 31, 235-40. (2000).
30. Theise, N. D. et al. Liver from bone marrow in humans. Hepatology 32, 11-6. (2000).
31. Cosenza, M. A., Zhao, M. L., Si, Q. & Lee, S. C. Human brain parenchymal microglia express CD14 and CD45 and are productively infected by HIV-1 in HIV-1 encephalitis. Brain Pathol 12, 442-55 (2002).
32. Meletis, K. & Risen, J. Have the bloody cells gone to our heads? J Cell Biol 155, 699-702. (2001).
33. Jiang, Y. et al. Pluripotency of mesenchymal stem cells derived from adult marrow. Nature 418, 41-49. (2002).
34. Krause, D. S. et al. Multi-organ, multi-lineage engraftment by a single bone marrow-derived stem cell. Cell 105, 369-77. (2001).
35. Kogler, G. et al. A new human somatic stem cell from placental cord blood with intrinsic pluripotent differentiation potential. J Exp Med 200, 123-35 (2004).
36. Erices, A., Conget, P. & Minguell, J. J. Mesenchymal progenitor cells in human umbilical cord blood. Br J Haematol 109, 235-42 (2000).
37. Erices, A. A., Allers, C. I., Conget, P. A., Rojas, C. V. & Minguell, J. J. Human cord blood-derived mesenchymal stem cells home and survive in the marrow of immunodeficient mice after systemic infusion. Cell Transplant 12, 555-61 (2003).
38. Mareschi, K. et al. Isolation of human mesenchymal stem cells: bone marrow versus umbilical cord blood. Haematologica 86, 1099-100 (2001).
39. Rubinstein, P. et al. Outcomes among 562 recipients of placental-blood transplants from unrelated donors [see comments]. N Engl J Med 339, 1565-77 (1998).
40. Yamashita, J. et al. Flk1-positive cells derived from embryonic stem cells serve as vascular progenitors. Nature 408, 92-6. (2000).
41. Bocci, G. et al. In-vitro evidence of autocrine secretion of vascular endothelial growth factor by endothelial cells from human placental blood vessels. Mol Hum Reprod 7, 771-7 (2001).
42. Shireman, P. K. & Quinones, M. P. Differential necrosis despite similar perfusion in mouse strains after ischemia. J Surg Res 129, 242-50 (2005).
43. Couffinhal, T. et al. Mouse model of angiogenesis. Am J Pathol 152, 1667-79 (1998).
44. Murohara, T. et al. Nitric oxide synthase modulates angiogenesis in response to tissue ischemia. J Clin Invest 101, 2567-78 (1998).
45. Pesce, M. et al. Myoendothelial differentiation of human umbilical cord blood-derived stem cells in ischemic limb tissues. Circ Res 93, e51-62 (2003).
46. Madlambayan, G. J. et al. Clinically relevant expansion of hematopoietic stem cells with conserved function in a single-use, closed-system bioprocess. Biol Blood Marrow Transplant 12, 1020-30 (2006).
47. Li, W. et al. High-resolution quantitative computed tomography demonstrating selective enhancement of medium-size collaterals by placental growth factor-1 in the mouse ischemic hindlimb Circulation 113, 2445-53 (2006).

1. A method of treating a patient with a disease involving endothelial cells, endothelial precursor cells, pericytes and/or muscle cells comprising administering to the patient cells selected from the group consisting of endothelial precursor cells, endothelial cells, pericytes and muscle cells obtained from multipotent CD45⁺HLA-ABC⁺Lin⁻ cells.

2. A method according to claim 1 comprising:
(a) culturing Lin^{neg} stem and progenitor cells under proliferation conditions to provide multipotent cells wherein the multipotent cells are CD45+HLA-ABC+ cells;
(b) culturing the multipotent cells under suitable differentiation conditions to produce a cell preparation comprising endothelial cells or muscle cells wherein the endothelial cells are characterized by expression of a member of the group consisting of CD31, CD133, von Willebrand factor, and VE-cadherin, and the muscle cells are characterized by expression of a member of the group consisting of MyoD, muscle specific actin, Ang-1, PDGF-β and myosin heavy chain; and
(c) administering multipotent cells of (a) or a cell preparation of (b) in an effective amount to the patient to treat the disease, wherein the disease is a peripheral vascular disease.

3. A method according to claim 2 wherein the mulitpotent cells comprise pericytes characterized by expression of a member of the group consisting of CD31, NG2 chondroitin sulphate proteoglycan, desmin, angiopoietin-1, osteonectin and Thy-1.

4. A method according to claim 2 wherein the multipotent cells comprise precursor endothelial cells characterized by expression of Flk-1.

5. A method according to claim 1, wherein the patient is a human.

6. A method according to claim 1, wherein the cells are administered to the patient by cell transplantation.

7. A method according to claim 1, wherein the multipotent cells are produced by culturing Lin^{neg} stem and progenitor cells isolated from umbilical cord blood in the presence of FGF-4, Flt-3 ligand and stem cell factor (SCF), and isolating the multipotent cells in the culture.

8. A method according to claim 1, wherein the disease is intermittent claudication or critical limb ischemia.

9. A purified cell preparation comprising a member of the group consisting of:
i. endothelial cells differentiated in vitro from multipotent CD45⁺HLA-ABC⁺Lin⁻ cells and characterized by the following properties: (a) CD31⁺; (b) CD133⁺; (c) Flk-1⁺; (d) elongated cells; (e) ability to grow into a network of vessel-like structures in vitro and in vivo; and (f) ability to secrete growth factors;
ii. muscle cells differentiated in vitro from multipotent CD45⁺HLA-ABC⁺Lin⁻ cells and characterized by the following properties: expression of MyoD, muscle actin, and/or myosin heavy chain, and ability to form vessels in vitro and in vivo;
iii. pericytes obtained by culturing Lin^{neg} stem and progenitor cells from umbilical cord blood in media comprising FGF-4, Flt-3 ligand and stem cell factor (SCF), wherein the pericytes are characterized by the expression of a member of the group consisting of CD31, NG2 chondroitin sulphate proteoglycan, desmin, angiopoietin-1, osteonectin. and Thy-1; and
iv. endothelial precursor cells obtained by culturing Lin^{neg} stem and progenitor cells from umbilical cord blood in media comprising FGF-4, Flt-3 ligand and stem cell factor (SCF) wherein the endothelial precursor cells are characterized by expression of Flk-1.

10. A method for producing a purified cell preparation as claimed in claim 9(i) comprising culturing multipotent CD45⁺HLA-ABC⁺Lin⁻ cells in endothelial differentiation media for at least one, two or three weeks.

11. (canceled)

12. A method for producing a purified cell preparation as claimed in claim 9(ii) comprising culturing multipotent CD45⁺HLA-ABC⁺Lin⁻ cells in muscle differentiation media for at least one, two or three weeks.

13-14. (canceled)

15. A method for obtaining cell preparations comprising cells selected from the group consisting of endothelial precursor cells, endothelial cells, pericytes, and muscle cells for autologous transplantation from a subject's own hematopoietic cells comprising
(a) obtaining hematopoietic cells from fresh or cryopreserved umbilical cord blood or bone marrow from a subject;
(b) separating out an enriched cell preparation comprising hematopoietic stem cells and hematopoietic progenitor cells which are Lin⁻;
(c) culturing the cells under proliferation conditions to produce multipotent CD45⁺HLA-ABC⁺ cells; and
(d) culturing the multipotent cells under suitable proliferation conditions or differentiation conditions to produce the cell preparations.

16. A method as claimed in claim 15 wherein the cell preparations comprise endothelial cells or muscle cells and the method comprises
(a) obtaining hematopoietic cells from fresh or cryopreserved umbilical cord blood from a subject;
(b) separating out an enriched cell preparation comprising Lin⁻ stem and progenitor cells;
(c) culturing the cells in medium comprising FGF4, SCF, and Flt-3 ligand, to produce multipotent CD45⁺HLA-ABC⁺ cells; and
(d) culturing the multipotent cells under suitable differentiation conditions to produce the cell preparation.

17. A method as claimed in claim 15 wherein the cell preparations comprise a member of the group consisting of pericytes and endothelial precursor cells, and the method comprises
(a) obtaining hematopoietic cells from fresh or cryopreserved umbilical cord blood or bone marrow from a subject;
(b) separating out an enriched cell preparation comprising Lin⁻ stem and progenitor cells; and
(c) culturing the cells in culture medium comprising FGF4, SCF, and Flt-3 ligand, to produce a cell preparation enriched for a member of the group consisting of pericytes and endothelial precursor cells.

18. A method as claimed in claim 15 further comprising administering the cell preparation to the subject.

19. A pharmaceutical composition comprising a cell preparation according to claim 9, and a pharmaceutically acceptable carrier, excipient, or diluent.

20. A method for assaying the activity of a test substance comprising the steps of:
(a) culturing multipotent CD45⁺HLA-ABC⁺Lin⁻ cells under suitable differentiation conditions in vitro to produce endothelial cells or muscle cells;
(b) exposing the cultured cells to a test substance; and
(c) detecting the presence or absence of an effect of the test substance on on a feature selected from the group consisting of the survival of the cells, a morphological characteristic, a functional characteristic, a physiological characteristic and a molecular biological property of the cells, whereby an effect altering the feature of the cells indicates the activity of the test substance.

21. A method of treating a peripheral vascular disease comprising administering to a subject with the disease a cell preparation according to claim 9 or a pharmaceutical composition comprising said cell preparation.

22. A kit for producing or using a cell preparation according to claim 9.

23. A kit for carrying out a method according to claim 1.
